(19) United States
(12) Patent Application Publication
(10) Pub. No.: US 2019/0374161 A1
(43) Pub. Date: Dec. 12, 2019

(54) EXOSUIT SYSTEMS AND METHODS FOR DETECTING AND ANALYZING LIFTING AND BENDING

(71) Applicant: SEISMIC HOLDINGS, INC., Menlo Park, CA (US)

(72) Inventors: Daniel Le Ly, Mountain View, CA (US); Ray Franklin Cowan, Mountain View, CA (US); Chung-Che Charles Wang, Palo Alto, CA (US); Andrew Robert Chang, Sunnyvale, CA (US)

(21) Appl. No.: 16/435,231

(22) Filed: Jun. 7, 2019

Related U.S. Application Data
(63) Continuation-in-part of application No. 16/256,812, filed on Jan. 24, 2019.
(60) Provisional application No. 62/682,179, filed on Jun. 8, 2018.

(51) Int. Cl. A61B 5/00; A61F 2/68; A61B 5/11; B25J 9/00

(52) U.S. Cl. CPC A61B 5/6811; A61F 2/68; A61B 5/1114; A61H 3/00; A61B 5/6812; A61B 5/1118; B25J 9/0006

(57) ABSTRACT

Systems and methods for detecting and analyzing lifting and bending motions performed by a user wearing an exosuit are discussed herein. Exosuits worn by users can monitor several movement factors that characterize the user's bending and lifting movements. The bend or lift is identified and analyzed, and feedback is provided to the user based on the analysis of the bend or lift.

### CROSS-REFERENCE TO RELATED APPLICATIONS

This application claims priority to U.S. Provisional Patent Application No. 62/682,179, filed Jun. 8, 2018, which is incorporated by reference in its entirety. This application is continuation-in-part of U.S. patent application Ser. No. 16/256,812, filed Jan. 24, 2019, which is incorporated by reference in its entirety

### TECHNICAL FIELD

This disclosure relates generally to the field of data analytics, and more specifically to systems and methods for detecting and analyzing lifting and bending.

### BACKGROUND

Wearable robotic systems have been developed for augmentation of humans' natural capabilities, or to replace functionality lost due to injury or illness. Sensors integrated into the wearable robotic can generating large volumes of new types of data, spurring a new revolution in data science and services. Some of this data can relate to bending and lifting.

### SUMMARY

Systems and methods for detecting and analyzing lifting and bending motions performed by a user wearing an exosuit are discussed herein. Exosuits worn by users can monitor several movement factors that characterize the user's bending and lifting movements. The bend or lift is identified and analyzed, and feedback is provided to the user based on the analysis of the bend or lift.

In one embodiment, an exosuit system is provided that can include an exosuit having a base layer, a power layer, and a plurality of sensors, wherein the exosuit is operative to provide the plurality of assistive movements; and control circuitry coupled to the power layer and the plurality of sensors. The control circuitry can be operative to receive data from the plurality of sensors during an exosuit use period; identify a lift event in the received sensor data; analyze data associated with the identified lift event to determine quality of the lift event; and provide feedback via the exosuit based on the determined quality of the lift event.

In another embodiment, an exosuit system is provided that includes an exosuit having a base layer, a power layer, and at least one sensor, wherein the exosuit is operative to provide the plurality of assistive movements; and control circuitry coupled to the power layer and the at least one sensor. The control circuitry is operative to receive data from the at least one sensor during an exosuit use period, wherein the at least one sensor provides three axes acceleration readings; identify a lift event in the received sensor data by analyzing the acceleration magnitudes; when a lift event is identified, analyze the pitch angles associated with the identified lift event to determine quality of the lift event; and provide feedback via the exosuit based on the determined quality of the lift event.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIGS. 1A-1C show front, back, and side views of a base layer of an exosuit according to an embodiment;

FIGS. 1D-1F show front, back, and side views, respectively, of a power layer according to an embodiment;

FIGS. 1G and 1H show respective front and back views of a human male's musculature anatomy, according to an embodiment;

FIGS. 1I and 1J show front and side views of an illustrative exosuit having several power layer segments that approximate many of the muscles shown in FIGS. 1G and 1H, according to various embodiments;

FIGS. 2A and 2B show front and back view of illustrative exosuit according to an embodiment;

FIG. 3 shows an illustrative symbiosis exosuit system according to an embodiment;

FIG. 4 shows illustrative process for implementing a symbiosis exosuit system according to an embodiment;

FIG. 5 shows illustrative displacement, velocity, and acceleration graphs corresponding to bending and lifting motion of the pictographic stickman according to an embodiment;

FIG. 6 shows an illustrative process for detecting and analyzing bends and lifts according to an embodiment;

FIG. 7 shows with more specificity an illustrative acceleration timing diagram showing the impulse of an exemplary lift action;

FIG. 8 shows an illustrative state machine for determining whether a lift or bend event has occurred when the sensor data is based on accelerometer only data;

FIG. 9 shows illustrative conditions according to an embodiment;

FIG. 10 shows illustrative pseudocode for analyzing the quality of the lift or bend according to an embodiment;

FIG. 11 shows an illustrative process for evaluating pitch angles from multiple sensors according to an embodiment;

FIGS. 12A and 12B show pitch angles obtained from lumbar, left, and right sensors during lifts considered unsafe and safe, according to an embodiment;

FIG. 13 shows an illustrative process for determining quality of a lift or bend based on only one 6-axis sensor, according to an embodiment;

FIGS. 14A and 14B show good and bad lifts, respectively, according to an embodiment;

FIG. 15 illustrates an example exosuit according to an embodiment; and

FIG. 16 is a schematic illustrating elements of an exosuit and a hierarchy of control or operating the exosuit according to an embodiment;

### DETAILED DESCRIPTION

In the following description, numerous specific details are set forth regarding the systems, methods and media of the disclosed subject matter and the environment in which such systems, methods and media may operate, etc., in order to provide a thorough understanding of the disclosed subject matter. It can be apparent to one skilled in the art, however, that the disclosed subject matter may be practiced without such specific details, and that certain features, which are well known in the art, are not described in detail in order to avoid complication of the disclosed subject matter. In addition, it can be understood that the examples provided below are exemplary, and that it is contemplated that there are other systems, methods and media that are within the scope of the disclosed subject matter.

Embodiments discussed herein use inertial sensing data obtained from sensors such as accelerometers, gyroscopes and magnetometers to measure and monitor the biomechanical bending and lifting actions of individuals. The inertial sensing equipment can be worn in discrete form-factors or blended into garments or incorporated into an exosuit, and embodiments discussed herein can use data provided by the sensors to determine and analyze bending and lifting actions, and provide appropriate feedback to the user on quality of the bending and lifting actions.

In the descriptions that follow, an exosuit or assistive exosuit is a suit that is worn by a wearer on the outside of his or her body. It may be worn under the wearer's normal clothing, over their clothing, between layers of clothing, or may be the wearer's primary clothing itself. The exosuit may be supportive, assistive, resistive, and/or enhancing as it physically interacts with the wearer while performing particular activities, or can provide other functionality such as communication to the wearer through physical expressions to the body, engagement of the environment, or capturing of information from the wearer. In some embodiments, a powered exosuit system can include several subsystems, or layers. In some embodiments, the powered exosuit system can include more or less subsystems or layers. The subsystems or layers can include the base layer, stability layer, power layer, sensor and controls layer, a covering layer, and user interface/user experience (UI/UX) layer.

The base layer provides the interfaces between the exosuit system and the wearer's body. The base layer may be adapted to be worn directly against the wearer's skin, between undergarments and outer layers of clothing, over outer layers of clothing or a combination thereof, or the base layer may be designed to be worn as primary clothing itself. In some embodiments, the base layer can be adapted to be both comfortable and unobtrusive, as well as to comfortably and efficiently transmit loads from the stability layer and power layer to the wearer's body in order to provide the desired assistance. The base layer can typically comprise several different material types to achieve these purposes. Elastic materials may provide compliance to conform to the wearer's body and allow for ranges of movement. The innermost layer is typically adapted to grip the wearer's skin, undergarments or clothing so that the base layer does not slip as loads are applied. Substantially inextensible materials may be used to transfer loads from the stability layer and power layer to the wearer's body. These materials may be substantially inextensible in one axis, yet flexible or extensible in other axes such that the load transmission is along preferred paths. The load transmission paths may be optimized to distribute the loads across regions of the wearer's body to minimize the forces felt by the wearer, while providing efficient load transfer with minimal loss and not causing the base layer to slip. Collectively, this load transmission configuration within the base layer may be referred to as a load distribution member. Load distribution members refer to flexible elements that distribute loads across a region of the wearer's body. Examples of load distribution members can be found in International Patent Publication No. WO 2016/138264, titled “Flexgrip,” the contents of which are incorporated herein by reference.

The load distribution members may incorporate one or more load lines or catenary curves to distribute loads across the wearer's body. Multiple load distribution members or catenary curves may be joined with pivot points, such that as loads are applied to the structure, the arrangement of the load distribution members pivots tightens or constricts on the body to increase the gripping strength. Compressive elements such as battens, rods, or stays may be used to transfer loads to different areas of the base layer for comfort or structural purposes. For example, a power layer component may terminate in the middle back due to its size and orientation requirements, however the load distribution members that anchor the power layer component may reside on the lower back. In this case, one or more compressive elements may transfer the load from the power layer component at the middle back to the load distribution member at the lower back.

The load distribution members may be constructed using multiple fabrication and textile application techniques. For example, the load distribution member can be constructed from a layered woven 45°/90° with bonded edge, spandex tooth, organza (poly) woven 45°/90° with bonded edge, organza (cotton/silk) woven 45°/90°, and Tyvek (non-woven). The load distribution member may be constructed using knit and lacing or horse hair and spandex tooth. The load distribution member may be constructed using channels and/or laces.

The base layer may include a flexible underlayer that is constructed to compress against a portion of the wearer's body, either directly to the skin, or to a clothing layer, and also provides a relatively high grip surface for one or more load distribution members to attach thereto. The load distribution members can be coupled to the underlayer to facilitate transmission of shears or other forces from the members, via the flexible underlayer, to skin of a body segment or to clothing worn over the body segment, to maintain the trajectories of the members relative to such a body segment, or to provide some other functionality. Such a flexible underlayer could have a flexibility and/or compliance that differs from that of the member (e.g., that is less than that of the members, at least in a direction along the members), such that the member can transmit forces along their length and evenly distribute shear forces and/or pressures, via the flexible underlayer, to skin of a body segment to which a flexible body harness is mounted.

Further, such a flexible underlayer can be configured to provide additional functionality. The material of the flexible underlayer could include anti-bacterial, anti-fungal, or other agents (e.g., silver nanoparticles) to prevent the growth of microorganisms. The flexible underlayer can be configured to manage the transport of heat and/or moisture (e.g., sweat) from a wearer to improve the comfort and efficiency of activity of the wearer. The flexible underlayer can include straps, seams, hook-and-loop fasteners, clasps, zippers, or other elements configured to maintain a specified relationship between elements of the load distribution members and aspects of a wearer's anatomy. The underlayer can additionally increase the ease with which a wearer can don and/or doff the flexible body harness and/or a system (e.g., a flexible exosuit system) or garment that includes the flexible body harness. The underlayer can additionally be configured to protect the wearer from ballistic weapons, sharp edges, shrapnel, or other environmental hazards (by including, e.g., panels or flexible elements of para-aramid or other high-strength materials).

The base layer can additionally include features such as size adjustments, openings and electro-mechanical integration features to improve ease of use and comfort for the wearer.

Size adjustment features permit the exosuit to be adjusted to the wearer's body. The size adjustments may allow the suit to be tightened or loosened about the length or circumference of the torso or limbs. The adjustments may comprise lacing, the Boa system, webbing, elastic, hook-and-loop or other fasteners. Size adjustment may be accomplished by the load distribution members themselves, as they constrict onto the wearer when loaded. In one example, the torso circumference may be tightened with corset-style lacing, the legs tightened with hook-and-loop in a double-back configuration, and the length and shoulder height adjusted with webbing and tension-lock fasteners such as cam-locks, D-rings or the like. The size adjustment features in the base layer may be actuated by the power layer to dynamically adjust the base layer to the wearer's body in different positions, in order to maintain consistent pressure and comfort for the wearer. For example, the base layer may be required to tighten on the thighs when standing, and loosen when sitting such that the base layer does not excessively constrict the thighs when seated. The dynamic size adjustment may be controlled by the sensor and controls layer, for example by detecting pressures or forces in the base layer and actuating the power layer to consistently attain the desired force or pressure. This feature does not necessarily cause the suit to provide physical assistance, but can create a more comfortable experience for the wearer, or allow the physical assistance elements of the suit to perform better or differently depending on the purpose of the movement assistance.

Opening features in the base layer may be provided to facilitate donning (putting the exosuit on) and doffing (taking the exosuit off) for the wearer. Opening features may comprise zippers, hook-and-loop, snaps, buttons or other textile fasteners. In one example, a front, central zipper provides an opening feature for the torso, while hook-and-loop fasteners provide opening features for the legs and shoulders. In this case, the hook-and-loop fasteners provide both opening and adjustment features. In other examples, the exosuit may simply have large openings, for example around the arms or neck, and elastic panels that allow the suit to be donned and doffed without specific closure mechanisms. A truncated load distribution member may be simply extended to tighten on the wearer's body. Openings may be provided to facilitate toileting so the user can keep the exosuit on, but only have to remove or open a relatively small portion to use the bathroom.

Electro-mechanical integration features attach components of the stability layer, power layer and sensor and controls layer into the base layer for integration into the exosuit. The integration features may be for mechanical, structural, comfort, protective or cosmetic purposes. Structural integration features anchor components of the other layers to the base layer. For the stability and power layers, the structural integration features provide for load-transmission to the base layer and load distribution members, and may accommodate specific degrees of freedom at the attachment point. For example, a snap or rivet anchoring a stability or power layer element may provide both load transmission to the base layer, as well as a pivoting degree of freedom. Stitched, adhesive, or bonded anchors may provide load transmission with or without the pivoting degree of freedom. A sliding anchor, for example along a sleeve or rail, may provide a translational degree of freedom. Anchors may be separable, such as with snaps, buckles, magnets, clasps, hooks, or any other suitable closure mechanism; or may be inseparable, such as with stitching, adhesives or other bonding. Size adjustment features as described above may allow adjustment and customization of the stability and power layers, for example to adjust the tension of spring or elastic elements in the passive layer, or to adjust the length of actuators in the power layer.

Other integration features such as loops, pockets, and mounting hardware may simply provide attachment to components that do not have significant load transmission requirements, such as batteries, circuit boards, sensors, or cables. Components that exist as gravitation weight can be transmitted into support grips, for example, the load line on the outseam. In some cases, components may be directly integrated into textile components of the base layer. For example, cables or connectors may include conductive elements that are directly woven, bonded or otherwise integrated into the base layer.

Electromechanical integration features may also protect or cosmetically hide components of the stability, power or sensor and controls layers. Elements of the stability layer (e.g. elastic bands or springs), power layer (e.g. flexible linear actuators or twisted string actuators) or sensor and controls layer (e.g. cables) may travel through sleeves, tubes, or channels integrated into the base layer, which can both conceal and protect these components. The sleeves, tubes, or channels may also permit motion of the component, for example during actuation of a power layer element. The sleeves, channels, or tubes may comprise resistance to collapse, ensuring that the component remains free and uninhibited within.

Enclosures, padding, fabric coverings, or the like may be used to further integrate components of other layers into the base layer for cosmetic, comfort, thermal regulation, or protective purposes. For example, components such as motors, batteries, cables, or circuit boards may be housed within an enclosure, fully or partially covered or surrounded in padded material such that the components do not cause discomfort to the wearer, are visually unobtrusive and integrated into the exosuit, and are protected from the environment. Opening and closing features may additionally provide access to these components for service, removal, or replacement.

In some cases—particularly for exosuits configurable for either provisional use or testing—a tether may allow for some electronic and mechanical components to be housed off the suit. In one example, electronics such as circuit boards and batteries may be over-sized, to allow for added configurability or data capture. If the large size of these components makes it undesirable to mount them on the exosuit, they could be located separately from the suit and connected via a physical or wireless tether. Larger, over-powered motors may be attached to the suit via flexible drive linkages that allow actuation of the power layer without requiring large motors to be attached to the suit. Such over-powered configurations allow optimization of exosuit parameters without constraints requiring all components to be attached or integrated into the exosuit.

Electro-mechanical integration features may also include wireless communication. For example, one or more power layer components may be placed at different locations on the exosuit. Rather than utilizing physical electrical connections to the sensors and controls layer, the sensor and controls layer may communicate with the one or more power layer components via wireless communication protocols such as Bluetooth, ZigBee, ultrawide band, or any other suitable communication protocol. This may reduce the electrical interconnections required within the suit. Each of the one or more power layer components may additionally incorporate a local battery such that each power layer component or group of power layer components are independently powered units that do not require direct electrical interconnections to other areas of the exosuit.

The stability layer provides passive mechanical stability and assistance to the wearer. The stability layer comprises one or more passive (non-powered) spring or elastic elements that generate forces or store energy to provide stability or assistance to the wearer. An elastic element can have an un-deformed, least-energy state. Deformation, e.g. elongation, of the elastic element stores energy and generates a force oriented to return the elastic element toward its least-energy state. For example, elastic elements approximating hip flexors and hip extensors may provide stability to the wearer in a standing position. As the wearer deviates from the standing position, the elastic elements are deformed, generating forces that stabilize the wearer and assist maintaining the standing position. In another example, as a wearer moves from a standing to seated posture, energy is stored in one or more elastic elements, generating a restorative force to assist the wearer when moving from the seated to standing position. Similar passive, elastic elements may be adapted to the torso or other areas of the limbs to provide positional stability or assistance moving to a position where the elastic elements are in their least-energy state.

Elastic elements of the stability layer may be integrated to parts of the base layer or be an integral part of the base layer. For example elastic fabrics containing spandex or similar materials may serve as a combination base/stability layer. Elastic elements may also include discrete components such as springs or segments of elastic material such as silicone or elastic webbing, anchored to the base layer for load transmission at discrete points, as described above.

The stability layer may be adjusted as described above, both to adapt to the wearer's size and individual anatomy, as well as to achieve a desired amount of pre-tension or slack in components of the stability layer in specific positions. For example, some wearers may prefer more pre-tension to provide additional stability in the standing posture, while others may prefer more slack, so that the passive layer does not interfere with other activities such as ambulation.

The stability layer may interface with the power layer to engage, disengage, or adjust the tension or slack in one or more elastic elements. In one example, when the wearer is in a standing position, the power layer may pre-tension one or more elastic elements of the stability layer to a desired amount for maintaining stability in that position. The pre-tension may be further adjusted by the power layer for different positions or activities. In some embodiments, the elastic elements of the stability layer should be able to generate at least 5 lbs force; preferably at least 50 lbs force when elongated.

The power layer can provide active, powered assistance to the wearer, as well as electromechanical clutching to maintain components of the power or stability layers in a desired position or tension. The power layer can include one or more flexible linear actuators (FLA). An FLA is a powered actuator capable of generating a tensile force between two attachment points, over a give stroke length. An FLA is flexible, such that it can follow a contour, for example around a body surface, and therefore the forces at the attachment points are not necessarily aligned. In some embodiments, one or more FLAs can include one or more twisted string actuators. In the descriptions that follow, FLA refers to a flexible linear actuator that exerts a tensile force, contracts or shortens when actuated. The FLA may be used in conjunction with a mechanical clutch that locks the tension force generated by the FLA in place so that the FLA motor does not have to consume power to maintain the desired tension force. Examples of such mechanical clutches are discussed below. In some embodiments, FLAs can include one or more twisted string actuators or flexdrives, as described in further detail in U.S. Pat. No. 9,266,233, titled “Exosuit System,” the contents of which are incorporated herein by reference. FLAs may also be used in connection with electrolaminate clutches, which are also described in the U.S. Pat. No. 9,266,233. The electrolaminate clutch (e.g., clutches configured to use electrostatic attraction to generate controllable forces between clutching elements) may provide power savings by locking a tension force without requiring the FLA to maintain the same force.

The powered actuators, or FLAs, are arranged on the base layer, connecting different points on the body, to generate forces for assistance with various activities. The arrangement can often approximate the wearer's muscles, in order to naturally mimic and assist the wearer's own capabilities. For example, one or more FLAs may connect the back of the torso to the back of the legs, thus approximating the wearer's hip extensor muscles. Actuators approximating the hip extensors may assist with activities such as standing from a seated position, sitting from a standing position, walking, or lifting. Similarly, one or more actuators may be arranged approximating other muscle groups, such as the hip flexors, spinal extensors, abdominal muscles or muscles of the arms or legs.

The one or more FLAs approximating a group of muscles are capable of generating at least 10 lbs over at least a ½ inch stroke length within 4 seconds. In some embodiments, one or more FLAs approximating a group of muscles may be capable of generating at least 250 lbs over a 6-inch stroke within ½ second. Multiple FLAs, arranged in series or parallel, may be used to approximate a single group of muscles, with the size, length, power, and strength of the FLAs optimized for the group of muscles and activities for which they are utilized.

The sensor and controls layer captures data from the suit and wearer, utilizes the sensor data and other commands to control the power layer based on the activity being performed, and provides suit and wearer data to the UX/UI layer for control and informational purposes.

Sensors such as encoders or potentiometers may measure the length and rotation of the FLAs, while force sensors measure the forces applied by the FLAs. Inertial measurement units (IMUs) measure and enable computation of kinematic data (positions, velocities and accelerations) of points on the suit and wearer. These data enable inverse dynamics calculations of kinetic information (forces, torques) of the suit and wearer. Electromyographic (EMG) sensors may detect the wearer's muscle activity in specific muscle groups. Electronic control systems (ECSs) on the suit may use parameters measured by the sensor layer to control the power layer. Data from the IMUs may indicate both the activity being performed, as well as the speed and intensity. For example, a pattern of IMU or EMG data may enable the ECS to detect that the wearer is walking at a specific pace. This information then enables the ECS, utilizing the sensor data, to control the power layer in order to provide the appropriate assistance to the wearer. Stretchable sensors may be used as a strain gauge to measure the strain of the elements in the stability layer, and thereby predict the forces in the elastic elements of the stability layer. Stretchable sensors may be embedded in the base layer or grip layer and used to measure the motion of the fabrics in the base layer and the motion of the body.

Data from the sensor layer may be further provided to the UX/UI layer, for feedback and information to the wearer, caregivers or service providers.

The UX/UI layer comprises the wearer's and others' interaction and experience with the exosuit system. This layer includes controls of the suit itself such as initiation of activities, as well as feedback to the wearer and caregivers. A retail or service experience may include steps of fitting, calibration, training and maintenance of the exosuit system. Other UX/UI features may include additional lifestyle features such as electronic security, identity protection and health status monitoring.

The assistive exosuit can have a user interface for the wearer to instruct the suit which activity is to be performed, as well as the timing of the activity. In one example, a user may manually instruct the exosuit to enter an activity mode via one or more buttons, a keypad, or a tethered device such as a mobile phone. In another example, the exosuit may detect initiation of an activity from the sensor and controls layer, as described previously. In yet another example, the user may speak a desired activity mode to the suit, which can interpret the spoken request to set the desired mode. The suit may be pre-programmed to perform the activity for a specific duration, until another command is received from the wearer, or until the suit detects that the wearer has ceased the activity. The suit may include cease activity features that, when activated, cause the suit to cease all activity. The cease activity features can take into account the motion being performed, and can disengage in a way that takes into account the user's position and motion, and safely returns the user to an unloaded state in a safe posture.

The exosuit may have a UX/UI controller that is defined as a node on another user device, such as a computer or mobile smart phone. The exosuit may also be the base for other accessories. For example, the exosuit may include a cell phone chip so that the suit may be capable of receiving both data and voice commands directly similar to a cell phone, and can communicate information and voice signals through such a node. The exosuit control architecture can be configured to allow for other devices to be added as accessories to the exosuit. For example, a video screen may be connected to the exosuit to show images that are related to the use of the suit. The exosuit may be used to interact with smart household devices such as door locks or can be used to turn on smart televisions and adjust channels and other settings. In these modes, the physical assist of the suit can be used to augment or create physical or haptic experiences for the wearer that are related to communication with these devices. For instance, an email could have a pat on the back as a form of physical emoji that when inserted in the email causes the suit to physically tap the wearer or perform some other type of physical expression to the user that adds emphasis to the written email.

The exosuit may provide visual, audio, or haptic feedback or cues to inform the user of various exosuit operations. For example, the exosuit may include vibration motors to provide haptic feedback. As a specific example, two haptic motors may be positioned near the front hip bones to inform the user of suit activity when performing a sit-to-stand assistive movement. In addition, two haptic motors may be positioned near the back hip bones to inform the user of suit activity when performing a stand-to-sit assistive movement. The exosuit may include one or more light emitting diodes (LEDs) to provide visual feedback or cues. For example, LEDS may be placed near the left and/or right shoulders within the peripheral vision of the user. The exosuit may include a speaker or buzzer to provide audio feedback or cues.

In other instances, the interaction of the FLA's with the body through the body harness and otherwise can be used as a form of haptic feedback to the wearer, where changes in the timing of the contraction of the FLA's can indicate certain information to the wearer. For instance, the number or strength of tugs of the FLA on the waist could indicate the amount of battery life remaining or that the suit has entered a ready state for an impending motion.

The control of the exosuit may also be linked to the sensors that are measuring the movement of the wearer, or other sensors, for instance on the suit of another person, or sensors in the environment. The motor commands described herein may all be activated or modified by this sensor information. In this example, the suit can exhibit its own reflexes such that the wearer, through intentional or unintentional motions, cues the motion profile of the suit. When sitting, for further example, the physical movement of leaning forward in the chair, as if to indicate an intention to stand up, can be sensed by the suit IMU's and be used to trigger the sit to stand motion profile. In one embodiment, the exosuit may include sensors (e.g., electroencephalograph (EEG) sensor) that are able to monitor brain activity may be used to detect a user's desire to perform a particular movement. For example, if the user is sitting down, the EEG sensor may sense the user's desire to stand up and cause the exosuit to prime itself to assist the user in a sit-to-stand assistive movement.

The suit may make sounds or provide other feedback, for instance through quick movements of the motors, as information to the user that the suit has received a command or to describe to the user that a particular motion profile can be applied. In the above reflex control example, the suit may provide a high pitch sound and/or a vibration to the wearer to indicate that it is about to start the movement. This information can help the user to be ready for the suit movements, improving performance and safety. Many types of cues are possible for all movements of the suit.

Control of the suit includes the use of machine learning techniques to measure movement performance across many instances of one or of many wearers of suits connected via the internet, where the calculation of the best control motion for optimizing performance and improving safety for any one user is based on the aggregate information in all or a subset of the wearers of the suit. The machine learning techniques can be used to provide user specific customization for exosuit assistive movements. For example, a particular user may have an abnormal gait (e.g., due to a car accident) and thus is unable to take even strides. The machine learning may detect this abnormal gait and compensate accordingly for it.

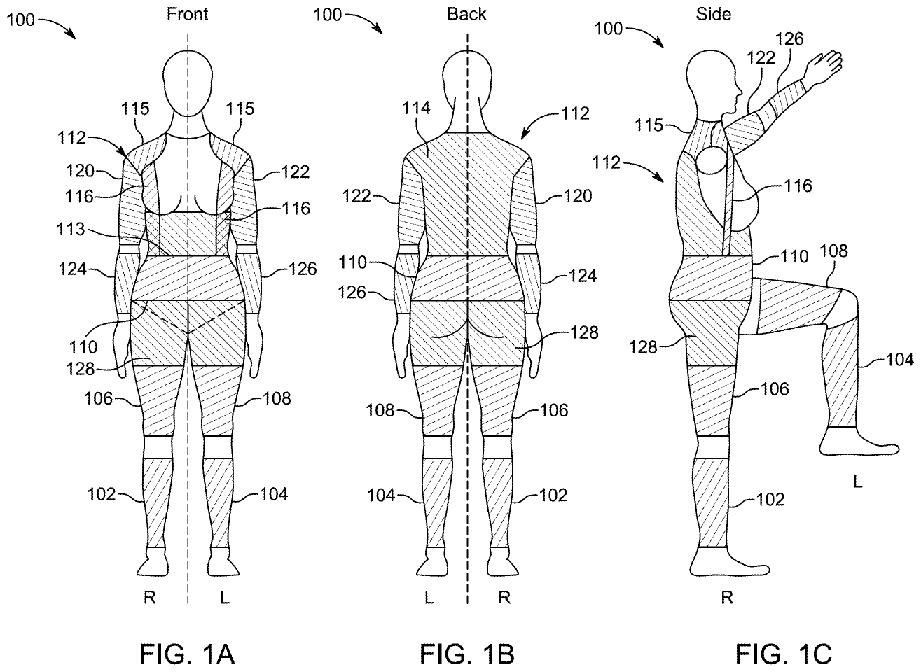

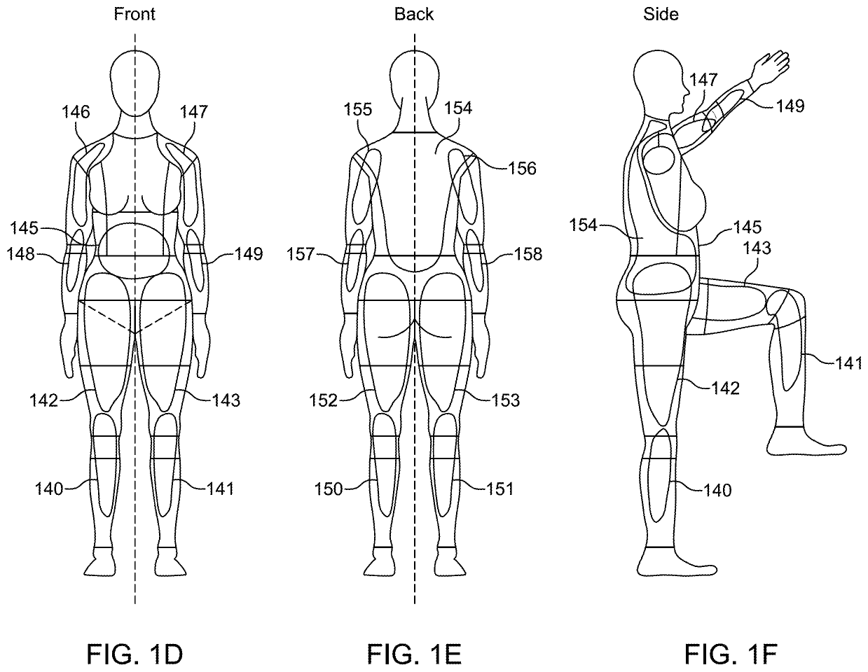

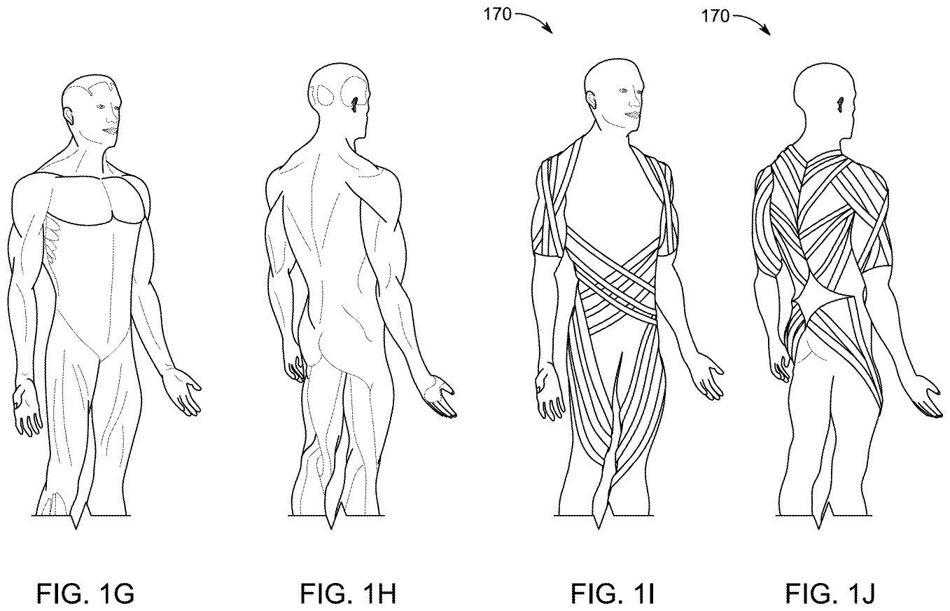

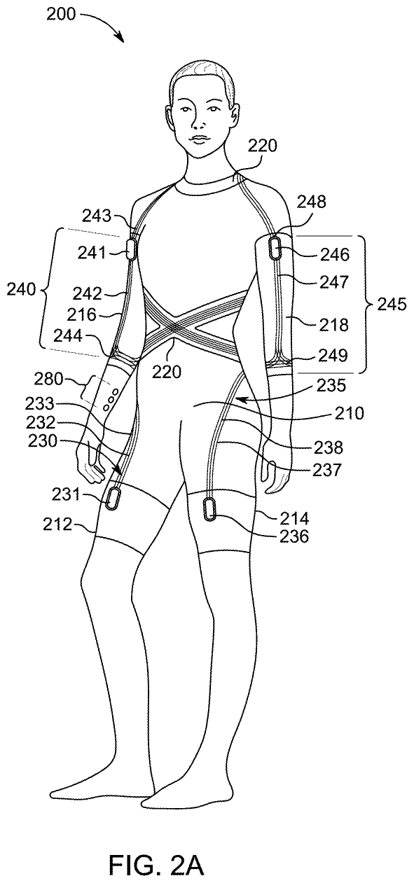

FIGS. 1A-IC show front, back, and side views of a base layer 100 of an exosuit according to an embodiment. Base layer 100 may be worn as a single piece or as multiple pieces. As shown, base layer 100 is shown to represent multiple pieces that can serve as load distribution members (LDMs) for the power layer (shown in FIGS. 1D-1F). Base layer 100 and any LDMs thereof can cover or occupy any part of the human body as desired. The LDMs shown in FIGS. 1A-IC are merely illustrative of a few potential locations and it should be appreciated that additional LDMs may be added or certain LDMs may be omitted.

Base layer 100 can include calf LDMs 102 and 104 that are secured around the calf region or lower leg portion of the human. Calf LDMs 102 and 104 are shown to be positioned between the knees and the ankles, but this is merely illustrative. If desired, calf LDM 102 and 104 can also cover the foot and ankle and/or the knee.

Base layer 100 can include thigh LDMs 106 and 108 that are secured around the thigh region of the human. Thigh LDMs 106 and 108 are shown to be positioned between the knees and an upper region of the thighs. In some embodiments, thigh LDMs 106 and 108 and calf LDMs 102 and 104, respectively, may be merged together to form leg LDMs that cover the entirety of the legs and/or feet.

Base layer 100 can include hip LDM 110 that is secured around a hip region of the human. LDM 110 may be bounded such that it remains positioned above the toileting regions of the human. Such bounding may make toileting relatively easy for the human as he or she would be not be required to remove base layer 100 to use the bathroom. In some embodiments, LDM 110 may be attached to thigh LDMs 106 and 108, but the toileting regions may remain uncovered. In another embodiment, a removable base layer portion may exist between LDM 100 and thigh LDMS 106 and 108.

Base layer 100 can include upper torso LDM 112 that is secured around an upper torso region of the human. Upper torso LDM 112 may include waist LDM 113, back LDM 114, shoulder LDM 115, and shoulder strap LDMs 116. Waist LDM 113, back LDM 114, shoulder LDM 115, and shoulder strap LDMs 116 may be integrally formed to yield upper torso LDM 112. In some embodiments, a chest LDM (not shown) may also be integrated into upper torso LDM 112. Female specific exosuits may have built in bust support for the chest LDM.

Base layer 100 can include upper arm LDMs 120 and 122 and lower arm LDMs 124 and 126. Upper arm LDMs 120 and 122 may be secured around bicep/triceps region of the arm and can occupy space between the shoulder and the elbow. Lower arm LDMs 124 and 126 may be secured around the forearm region of the arm and can occupy the space between the elbow and the wrist. If desired, upper arm LDM 120 and lower arm LDM 124 may be integrated to form an arm LDM, and upper arm LDM 122 and lower arm LDM 126 may be integrated to form another arm LDM. In some embodiments, arm LDMS 120, 122, 124, and 126 may form part of upper torso LDM 112.

Base layer 100 can include gluteal/pelvic LDM 128 that is secured the gluteal and pelvic region of the human. LDM 128 may be positioned between thigh LDMs 106 and 108 and hip LDM 110. LDM 128 may have removable portions such as buttoned or zippered flaps that permit toileting. Although not shown in FIGS. 1A-IC, LDMs may exist for the feet, toes, neck, head, hands, fingers, elbows, or any other suitable body part.

As explained above, the LDMs may serve as attachment points for components of the power layer. In particular, the components that provide muscle assistance movements typically need to be secured in at least two locations on the body. This way, when the flexible linear actuators are engaged, the contraction of the actuator can apply a force between the at least two locations on the body. With LDMs strategically placed around the body, the power layer can also be strategically placed thereon to provide any number of muscle assistance movements. For example, the power layer may be distributed across different LDMs or within different regions of the same LDM to approximate any number of different muscles or muscle groups. The power layer may approximate muscle groups such as the abdominals, adductors, dorsal muscles, shoulders, arm extensors, wrist extensors, gluteals, arm flexors, wrist flexors, scapulae fixers, thigh flexors, lumbar muscles, surae, pectorals, quadriceps, and trapezii. The power layer may also apply forces along paths that are not representative of biological muscle groups. For example, the power layer may wrap around the knee for stability and support.

FIGS. 1D-IF show front, back, and side views, respectively, of a power layer according to an embodiment. The power layer is shown as multiple segments distributed across and within the various LDMs. As shown, the power layer can include power layer segments 140-158. Each of power layer segments can include any number of flexible linear actuators. Some of the power layer segments may exist solely on the anterior side of the body, exist solely on the posterior side, start on the anterior side and wrap around to the posterior side, start on the posterior side and wrap around to the anterior side, or wrap completely around a portion of the body. Power layer segment (PLS) 140 may be secured to LDM 102 and LDM 106, and PLS 141 may be secured to LDM 104 and LDM 108. PLS 142 may be secured to LDM 106 and LDM 110 and/or LDM 114, and PLS 143 may be secured to LDM 108 and LDM 110 and/or LDM 114. PLS 145 may be secured to LDM 110 and LDM 113 and/or to LDM 114 or LDM 128. PLS 146 may be secured to LDM 115 and LDM 120, and PLS 147 may be secured to LDM 115 and LDM 122. PLS 148 may be secured to LDM 120 and LDM 124, and PLS 149 may be secured to LDM 122 and LDM 126.

PLS 150 may be secured to LDM 104 and LDM 108, and PLS 151 may be secured to LDM 102 and LDM 106. PLS 152 may be secured to LDM 106 and LDM 110 and/or to LDM 113, and PLS 153 may be secured to LDM 108 and LDM 110 and/or LDM 113. PLS 154 may be secured to LDM 112 and LDM 110. PLS 155 may be secured to LDM 112 and LDM 120, and PLS 156 may be secured to LDM 112 and LDM 122. PLS 157 may be secured to LDM 120 and LDM 124, and PLS 158 may be secured to LDM 122 and LDM 126.

It should be appreciated that the power layer segments are merely illustrative and that additional power layer segments may be added or that some segments may be omitted. In addition, the attachment points for the power layer segments are merely illustrative and that other attachment points may be used.

The human body has many muscles, including large and small muscles that are arranged in all sorts of different configuration. For example, FIGS. 1G and 1H show respective front and back views of a human male's musculature anatomy, which shows many muscles. In particular, the abdominals, adductors, dorsal muscles, shoulders, arm extensors, wrist extensors, gluteals, arm flexors, wrist flexors, scapulae fixers, thigh flexors, lumbar muscles, pectorals, quadriceps, and trapezii are all shown. It should be understood that several muscles and tendons are not shown.

The LDMs may be designed so that they can accommodate different sizes of individuals who don the exosuit. For example, the LDMs may be adjusted to achieve the best fit. In addition the LDMs are designed such that the location of the end points and the lines of action are co-located with the bone structure of the user in such a way that the flexdrive placement on the exosuit system are aligned with the actual muscle structure of the wearer for comfort, and the moment arms and forces generated by the flexdrive/exosuit system feel aligned with the forces generated by the wearer's own muscles.

FIGS. 1I and 1J show front and side views of illustrative exosuit 170 having several power layer segments that approximate many of the muscles shown in FIGS. 1G and 1H. The power layer segments are represented by the individual lines that span different parts of the body. These lines may represent specific flexible linear actuators or groups thereof that work together to form the power layer segments that are secured to the LDMs (not shown). As shown, the FLAs may be arrayed to replicate at least a portion of each of the abdominal muscles, dorsal muscles, shoulder muscles, arm extensor and flexor muscles, gluteal muscles, quadriceps muscles, thigh flexor muscles, and trapezii muscles. Thus, exosuit 170 exemplifies one of many possible different power layer segment arrangements that may be used in exosuits in accordance with embodiments discussed herein. These power layer segments are arranged so that the moment arms and forces generated feel like forces being generated by the user's own muscles, tendons, and skeletal structure. It should be appreciated that the power layer segments can be designed to approximate other muscles and tendons that are not shown in FIGS. 1G and 1H. Other possible power layer segment arrangements are illustrated and discussed below.

The power layer segments may be arranged such that they include opposing pairs or groups, similar to the way human muscles are arranged in opposing pairs or groups of muscles. That is, for a particular movement, the opposing pairs or groups can include protagonist and antagonist muscles. While performing the movement, protagonist muscles may perform the work, whereas the antagonist muscles provide stabilization and resistance to the movement. As a specific example, when a user is performing a curl, the biceps muscles may serve as the protagonist muscles and the triceps muscles may serve as the antagonist muscles. In this example, the power layer segments of an exosuit may emulate the biceps and triceps. When the biceps human muscle is pulling to bend the elbow, the exosuit triceps power layer segment can pull on the other side of the joint to resist bending of the elbow by attempting to extend it. The power layer segment can be, for example, either be a FLA operating alone to apply the force and motion, or a FLA in series with an elastic element. In the latter case, the human biceps would be working against the elastic element, with the FLA adjusting the length and thereby the resistive force of the elastic element.

Thus, by arranging the power layer segments in protagonist and antagonist pairs, the power layers segments can mimic or emulate any protagonist and antagonist pairs of the human anatomy musculature system. This can be used to enable exosuits to provide assistive movements, alignment movements, and resistive movements. For example, for any exercise movement requires activation of protagonist muscles, a subset of the power layer segments can emulate activation of antagonist muscles associated with that exercise movement to provide resistance.

The design flexibility of the LDMs and PLSs can enable exosuits to be constructed in accordance with embodiments discussed herein. Using exosuits, the power layer segments can be used to resist motion, assist motion, or align the user's form.

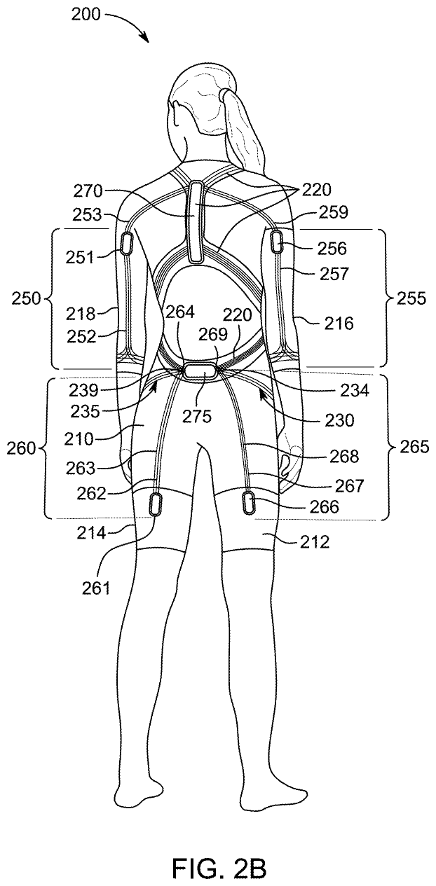

FIGS. 2A and 2B show front and back view of illustrative exosuit 200 according to an embodiment. Exosuit 200 may embody some or all of the base layer, stability layer, power layer, sensor and controls layer, a covering layer, and user interface/user experience (UI/UX) layer, as discussed above. In addition, exosuit 200 may represent one of many different specification implementations of the exosuit shown in FIGS. 1A-1F. Exosuit 200 can include base layer 210 with thigh LDMs 212 and 214, arm LDMs 216 and 218, and upper torso LDM 202. Thigh LDMs 212 and 214 may wrap around the thigh region of the human, and arm LDMs 216 and 218 may wrap around arm region (including the elbow) of the human. Upper torso LDM 220 may wrap around the torso and neck of the human as shown. In particular, LDM 220 may cross near the abdomen, abut the sacrum, cover a portion of the back, and extend around the neck.

Exosuit 200 can include PLSs 230 and 235 secured to thigh LDM 212 and 214 and upper torso LDM 220. PLSs 230 and 235 may provide leg muscle flexor movements. PLS 230 may include flexdrive subsystem 231, twisted string 232, and power/communication lines 233. Flexdrive subsystem 231 may include a motor, sensors, a battery, communications circuitry, and/or control circuitry. Twisted string 232 may be attached to flexdrive subsystem 231 and an attachment point 234 on LDM 220. Power/communications lines 233 may convey control signals and/or power to flexdrive subsystem 231. PLS 235 may include flexdrive subsystem 236, twisted string 237, and power/communication lines 238. Twisted string 237 may be attached to flexdrive subsystem 236 and attachment point 239.

Exosuit 200 can include PLSs 240 and 245 and PLSs 250 and 255 that are secured to LDMs 216, 218, and 220 (as shown). PLSs 240 and 245 may provide arm muscle flexor movements, and PLSs 250 and 255 may provide arm muscle extensor movements. PLS 240 may include flexdrive subsystem 241, twisted string 242, and power/communication lines 243. Twisted string 242 may be attached to flexdrive subsystem 241 and attachment point 244. Power/communication lines 243 may be coupled to power and communications module 270. PLS 245 may include flexdrive subsystem 246, twisted string 247, and power/communication lines 248. Twisted string 247 may be attached to flexdrive subsystem 246 and attachment point 249. Power/communication lines 248 may be coupled to power and communications module 270. PLS 250 may include flexdrive subsystem 251, twisted string 252, and power/communication lines 253. Twisted string 252 may be attached to flexdrive subsystem 251 and attachment point 254. Power/communication lines 253 may be coupled to power and communications module 270. PLS 250 may include flexdrive subsystem 256, twisted string 257, and power/communication lines 258. Twisted string 256 may be attached to flexdrive subsystem 256 and attachment point 259. Power/communication lines 258 may be coupled to power and communications module 270.

Exosuit 200 can include PLS 260 and 265 that are secured to thigh LDMs 212 and 214 and LDM 220. PLSs 260 and 265 may provide leg muscle flexor movements. PLS 260 may include flexdrive subsystem 261, twisted string 262, and power/communication lines 263. Twisted string 262 may be attached to flexdrive subsystem 261 and attachment point 264. Power/communication lines 263 may be coupled to power and communications module 275. PLS 266 may include flexdrive subsystem 266, twisted string 267, and power/communication lines 268. Twisted string 267 may be attached to flexdrive subsystem 266 and attachment point 269. Power/communication lines 263 may be coupled to power and communications module 275

Exosuit 200 is designed to assist, resist, align, or enhance movements being performed by the user of the suit. Exosuit 200 may include many sensors in various locations to provide data required by control circuitry to provide such movements. These sensors may be located anywhere on base layer 210 and be electrically coupled to power and communications lines (e.g., 233, 237, 243, 247, 253, 257, 263, 267, or other lines). The sensors may provide absolute position data, relative position data, accelerometer data, gyroscopic data, inertial moment data, strain gauge data, resistance data, or any other suitable data.

Exosuit 200 may include user interface 280 that enables the user to control the exosuit. For example, user interface 280 can include several buttons or a touch screen interface. User interface 280 may also include a microphone to receive user spoken commands. User interface 280 may also include a speaker that can be used to playback voice recordings. Other user interface element such as buzzers (e.g., vibrating elements) may be strategically positioned around exosuit 200.

Exosuit 200 can include communications circuitry such as that contained in power and communications module 270 or 275 to communicate directly with a user device (e.g., a smartphone) or with the user device via a central server. The user may use the user device to select one or more movements he or she would like to perform, and upon selection of the one or more movements, exosuit 200 can the assist, resist, or align movement. The user device or exosuit 200 may provide real-time alignment guidance as to the user's performance of the movement, and exosuit 200 may provide resistance, alignment, or assistance to the movement.

An exosuit can be operated by electronic controllers disposed on or within the exosuit or in wireless or wired communication with the exosuit. The electronic controllers can be configured in a variety of ways to operate the exosuit and to enable functions of the exosuit. The electronic controllers can access and execute computer-readable programs that are stored in elements of the exosuit or in other systems that are in direct or indirect communications with the exosuit. The computer-readable programs can describe methods for operating the exosuit or can describe other operations relating to a exosuit or to a wearer of a exosuit.

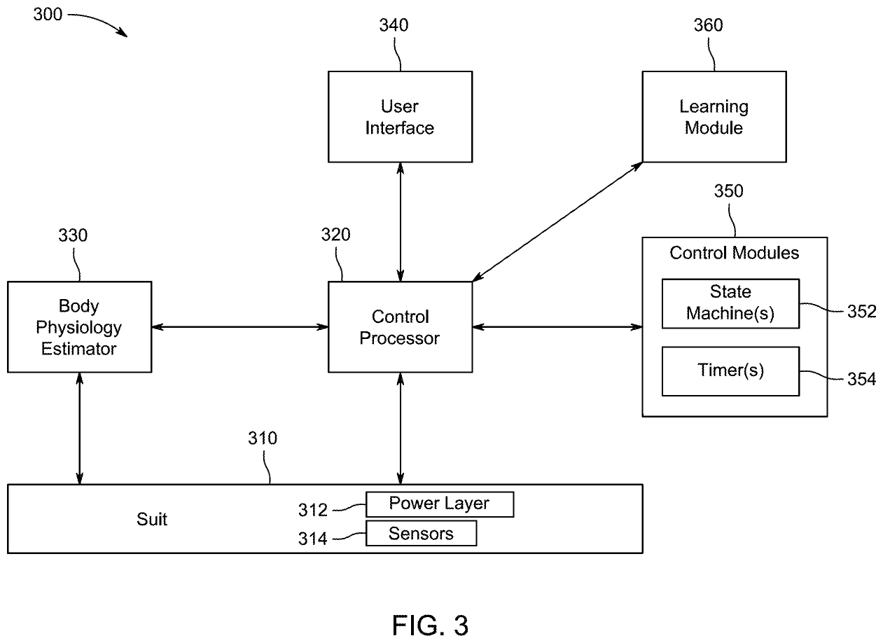

FIG. 3 shows an illustrative symbiosis exosuit system 300 according to an embodiment. The symbiosis enables the exosuit to serve as an autonomous exosuit nervous system that mimics or emulates the nervous system of a lifeform such as a human being. That is, a nervous system is responsible for basic life functions (e.g., breathing, converting food into energy, and maintaining muscle balance) that are performed automatically without requiring conscious thought or input. The autonomous exosuit nervous system enables the exosuit to automatically provide assistance to the user when and where the user needs it without requiring intervention by the user. Exosuit system 300 can do this by tracking the user's body physiology and automatically controlling the suit to provide the anticipated or required support and/or assistance. For example, if a user has been standing for a prolonged period of time, one or more of the muscles being used to help the user stand may begin to tire, and a result, the user's body may exhibit signs of fatigue. Exosuit 300 can observe this muscle fatigue (e.g., due to observed physiological signs) and can automatically cause exosuit 300 to engage the appropriate power layers to compensate for the muscle fatigue.

Symbiosis of exosuit 300 may be expressed in different autonomy levels, where each autonomy level represents a degree to which physiological factors are observed and a degree to which suit assistance or movement actions are performed based on the observed physiological factors. For example, the symbiosis levels can range from a zero level of autonomy to absolute full level of autonomy, with one or more intermediate levels of autonomy. As metaphorical example, autonomous cars operate according to different levels, where each level represents a different ability for the car to self-drive. The symbiosis levels of exosuit operation can be stratified in a similar manner. In a zero level of autonomy, exosuit 300 may not monitor for any physiological cues, nor automatically engage any suit assistance or movement actions. Thus, in a zero level, the user may be required to provide user input to instruct the suit to perform a desired movement or assistance. In an absolute full level of autonomy, exosuit 300 may be able to observe and accurately analyze the observed physiological data (e.g., with 99 percent accuracy or more) and automatically execute the suit assistance or movement actions in a way expressly desired by the user. Thus, in the absolute full level, the exosuit seamlessly serves as an extension of the user's nervous system by automatically determining what the user needs and providing it.

The one or more intermediate levels of autonomy provide different observable physiological results that are accurate but do not represent the absolute nature of the absolute full level of autonomy. For example, the intermediate levels may represent that the exosuit is fully capable of autonomously performing certain actions (e.g., sit to stand) but not others. A corollary to this is ABS braking; the ABS braking system automatically figures out how best to stop the vehicle without requiring the user to pump the brakes or engage in any other activity other than stepping on the brake pedal. In the exosuit context, the exosuit knows when the user wishes to stand from a sitting position, the exosuit knows when the user wishes to perform the movement and engages the appropriate power layer segments to assist in the movement. The intermediate levels may also exist while the exosuit is learning about its user. Each user is different, and the physiological responses are therefore different and particular to each user. Therefore, the ability to discern the physiological cues and the assistance and movements made in response thereto may endure a learning curve before the suit is able to operate at the absolute full level.

FIG. 3 shows that exosuit system 300 can include suit 310, control processor 320, body physiology estimator 330, user interface 340, control modules 350, and learning module 360. Suit 310 can be any suitable exosuit (e.g., exosuit 200) and can include, among other things, power layer 312 and sensors 314. Control processor 320 may process instructions, pass data, and control the suit. Control processor 320 may be connected to suit 310, body physiology estimator 330, user interface 340, control modules 350, and learning module 360. Control processor 320 may provide signals to suit 310 to control, for example, operation of power layer 312.

Body physiology estimator 330 may receive data inputs from sensor 314, control processor 320, and other components if desired. Estimator 330 is operative to analyze the data to ascertain the physiology of the user. Estimator 330 may apply data analytics and statistics to the data to resolve physiological conditions of the user's body. For example, estimator 330 can determine whether the user is sitting, standing, leaning, laying down, laying down on a side, walking, running, jumping, performing exercise movements, playing sports, reaching, holding an object or objects, or performing any other static or active physiological event. The results may be provided to control modules 350, for example, via control processor 320.

Sensors 314 can include an accelerometer, gyroscope, magnetometer, altimeter sensor, EKG sensor, and any other suitable sensor. Sensors 314 may be integrated anywhere within the exosuit, though certain locations may be more preferred than others. The sensor can be placed near the waist, upper body, shoes, thigh, arms, wrists or head. In some embodiments, sensors can be embedded onto the equipment being used by the user. In some embodiments, the sensors can be contained external to the exosuit. For example, if worn on the wrist or arm of a worker, the device can be embedded into a watch, wrist band, elbow sleeve, or arm band. A second device may be used and clipped on the waist on the pelvis, or slipped into a pocket in the garment, embedded into the garment itself, back-brace, belt, hard hat, protective glasses or other personal protective equipment the worker is wearing. The device can also be an adhesive patch worn on the skin. Other form factors can also clip onto the shoe or embedded into a pair of socks or the shoe itself.

Control modules 350 can include various state machines 352 and timers 354 operative to control operation of suit 310 based on outputs supplied by estimator 330, inputs received via user interface 340, and signals provided by control processor 320. Multiple state machines 352 may control operation of the suit. For example, a master state machine may be supported by multiple slave state machines. The slave state machines may be executed in response to a call from the master state machine. In addition, the slave state machines may execute specific assistance functions or movements. For example, each of a sit-to-stand assistance movement, stand-to sit movement, stretch movement, standing movement, walking movement, running movement, jumping movement, crouch movement, specific exercise movement, or any other movement may have its own slave state machine to control suit operation.

Learning module 360 may be operative to learn preferences, peculiarities, or other unique features of a particular user and feedback the learnings to body physiology estimator 330 and control module 350. In some embodiments, learning module 360 may use data analytics to learn about the user. For example, learning module 360 may learn that a particular user walks with a particular gait and cadence. The gait and cadence learnings can be used to modify state machines 352 that control walking for that user. In another embodiment, learning module 360 may incorporate user feedback received via user interface 340. For example, a user may go through an initial setup process whereby the user is instructed to perform a battery of movements and provide responses thereto so that state machines 352 and timers 354 are set to operate in accordance with the preferences of the user.

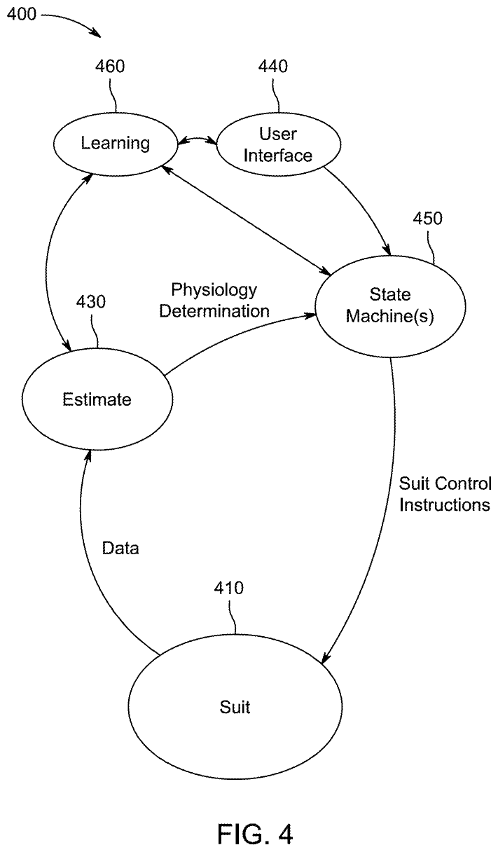

FIG. 4 shows illustrative process 400 for implementing symbiosis exosuit system 300 according to an embodiment. Process 400 includes suite 410, estimator 430, user interface 440, and state machines 450. Process 400 can be represented by a continuous feedback loop in which data is supplied from suit 410 to estimator 430, which provides a physiology determination to state machines 450, which uses the determination to generate suit control instructions that are provided to suit 410. User inputs received via user interface 440 may provide user specified controls that can instruct state machines 450 to execute a particular movement. The autonomous exosuit nervous system is implemented through the continuous feedback loop. The continuous feedback loop enables the autonomous exosuit nervous system to provide rapid response and control of exosuit 410. For example, if the user is sitting down, the estimator 430 can determine that the sitting position is the current physiological determination. Assume that the user reaches for something on a table. Such a movement may result in a movement that appears to be a sit-to-stand. In response to this movement, estimator 430 may register it as the start of a sit-to-stand physiological determination and instruct state machines 450 to initiate a sit-to-stand movement. This way, regardless of whether the user actually stands or sits back down, suit 410 is primed and ready to immediately perform the assistance movement. Further assume that the user sits back down (after having grabbed the item on the table). In response to initiation of the sit down movement, estimator 430 can make this determination as it is happening and instruct state machines 450 to cease the sit-to-stand operation. Thus, the continuous feedback loop provides real-time assessment and instantaneous suit controls in response to the user's immediate physiological needs, and not after.

In some embodiments, estimator 430 may be able to determine that the user is was attempting to reach something on the table while also performing the motion that includes at least the start of a sit to stand movement. Estimator 430 may be able to correlate the reaching motion with the sit-to-stand motion and decide that the user does not actually need to stand, but may require an appropriate amount of assist to reach the item. In this particular situation, state machine 450 may activate a power layer segment (e.g., a particular one of the hip extensors) to provide the user with the reach assistance.

Learning 460 can receive and provide data to estimator 430, user interface 440, and state machines 450. Learning 460 may be leveraged to update state machines 450 and/or estimator 430.

When a user wears an exosuit outfitted with one or more sensors, the sensor can detect motion and wake up from a low power sleep mode. When the user begins walking, for example, the sensor and system will recognize a walking signal and then begins an auto-calibration sequence that generates a reference orientation frame to abstract the sensor placement (and corresponding raw sensor data) from additional computation. Such calibration is required when the sensor is an accelerometer only sensor (e.g., 3-axis sensor) and such calibration is not necessary for gyroscope+accelerometer sensors (e.g., 6-axis sensors).

A method for posture detection and feedback system is described in U.S. Pat. No. 8,928,484, titled “SYSTEM AND METHOD FOR BIOMECHANICAL POSTURE DETECTION AND FEEDBACK”, filed 12 Jul. 2013, the disclosure of which is incorporated by reference in its entirety.

The exosuit may autocalibrate its sensors each time one or more of the sensors detect motion or wakes up from a low power sleep mode. When the user begins walking, for example, the sensor and system can recognize a walking signal and then begins an auto-calibration sequence that generates a reference orientation frame to abstract sensor placement (and corresponding raw sensor data) from additional computation. A method for auto calibration is described in US Patent Publication no. 2017/0258374, titled “SYSTEM AND METHOD FOR AUTOMATIC POSTURE CALIBRATION”, which is hereby incorporated in its entirety by this reference.

In addition to auto-calibration, all sensors that are part of the exosuit can be synchronized to the same time. This can be accomplished by connecting with each other, being connected via an onboard processor system, or with a peripheral device such as a smart phone or smart watch that has a reliable real-time clock.

After the auto-calibration sequence, the exosuit may determine the location of where each sensor is being worn on the body by analyzing unique location specific motion signatures. A method for automatic location detection has been described in US Patent Publication no. 2018/0264320, titled “SYSTEM AND METHOD FOR AUTOMATIC WEARABLE SENSOR LOCATION DETECTION FOR WEARABLE SENSORS,” which is hereby incorporated in its entirety. When the sensor locations are determined (e.g., the specific locations and orientation of the sensors on the exosuit), the exosuit system can determine the appropriate location specific posture model. For example, the sensor may determine that the sensor is being worn on the clavicle and run a posture model for each person each time the person dons the exosuit.

In a separate example, a smart phone application or base station can communicate with the sensor(s) to explicitly configure the exosuit's location or application model (i.e., posture vs lifting and bending). Another instance, the sensor may be programmed with both a posture and daily activities model and a lifting and bending model. When a user walks, the sensor and system analyzes their walking gait, when the user is sitting or standing, the sensor and system can analyze their posture, and when a user bends down to lift an object, the sensor and system can detect, analyze and provide feedback.

After calibration, standard computations can be performed from the calibrated sensor data to compute a user's activity state, posture state and detect a lifting or bending transition. If a lifting or bending transition is detected, then the sensor system can compute parameters associated with the transition.

If a bending event is detected, parameters such as maximum bending angle, speed of angle change, and forces associated with the bending event can be computed and real-time feedback can be provided to remind a user to bend correctly.

If a lifting event is detected, parameters such as vertical, lateral and forward displacement, low-back angle, angular rotation about the sagittal, coronal or transverse planes, impulse acceleration, and other relevant forces associated with a lifting event can be computed. Data can be logged per event and synced with a software application or cloud database. Real-time feedback can be provided to the user if the user was bending or rotating significantly above a specific threshold.

Detecting lifting and bending events can include identifying and marking a variety of signatures in time-series data from inertial measurement sensors (e.g., accelerometers, gyroscopes, and magnetometers). Event detection with high sensitivity and specificity is a critical first step towards evaluating biomechanic form as it provides a method to isolate the relevant time-series data for analysis. There are a variety of different approaches to detecting lifting and bending events, and selecting the appropriate approach often depends on where the sensors are located and which types are available. In one implementation, the sensors can be located on the torso (e.g., back) and on both legs, where the motion is accentuated due to the large range of motion of the upper body during a lifting or bending event.

There can be three general classes of sensors available for use in detecting and analyzing bending and lifting movements. The class of sensors determines which algorithms are used to analyze the bending and lifting movements. A first class of sensors includes only one or more 3-axis accelerometers. A second class of sensors includes at least three 6-axis inertial motion units (IMUs), where each IMU includes a three axis gyroscope and a three axis accelerometer. A third class of sensor includes only one 6-axis IMU. A fourth class of sensors can include one or more 9-axis IMUs (e.g., an IMU that include a gyroscope, an accelerometer, and a magnetometer).

If only the accelerometer sensor is available, detecting lifting and bending events can be challenging as the reference frame of the human body cannot be determined and the gravitational force cannot be isolated from other dynamic forces. Without the ability to separate which accelerations are due to gravity and which accelerations are due to motion, a general approach is required. In one approach, the accelerometer magnitude can be segmented by detecting a specific signature that is indicative of lifting and bending, particularly by detecting a specific sequence of impulse-like signatures. The vertical displacement of the upper body follows a relatively universal signature for all lifting or bending events. If the motion is approximated as purely vertical, then the sensor follows a specific displacement curve. By differentiating the motion, the corresponding velocity and acceleration curves can be obtained and it is clear that the acceleration curve is characterized by a sequence of three impulse-like signatures.

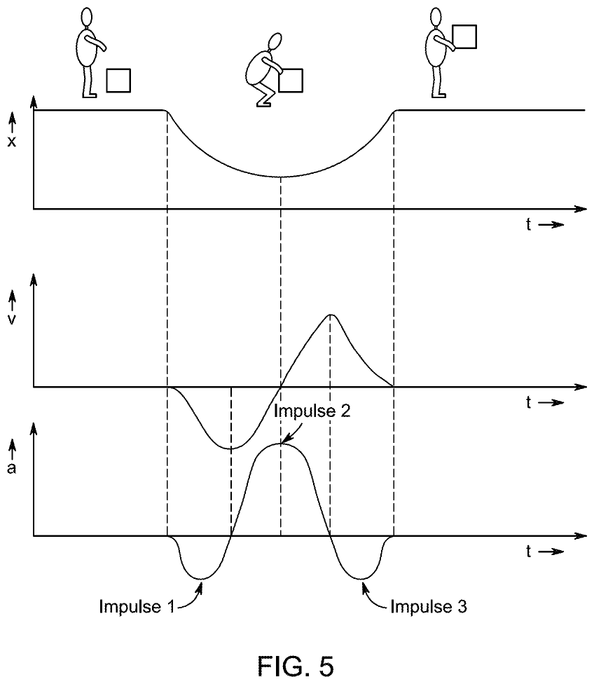

FIG. 5 shows illustrative displacement, velocity, and acceleration graphs corresponding to bending and lifting motion of the pictographic stickman according to an embodiment. Three impulse-like signatures are shown in the acceleration graph. Referring to the acceleration graph, the acceleration is near zero when the user is standing, but as the bends down to perform the lift action, the acceleration reaches a first low magnitude, and then proceeds to a high magnitude, and goes back down to a second low magnitude before settling back to zero.

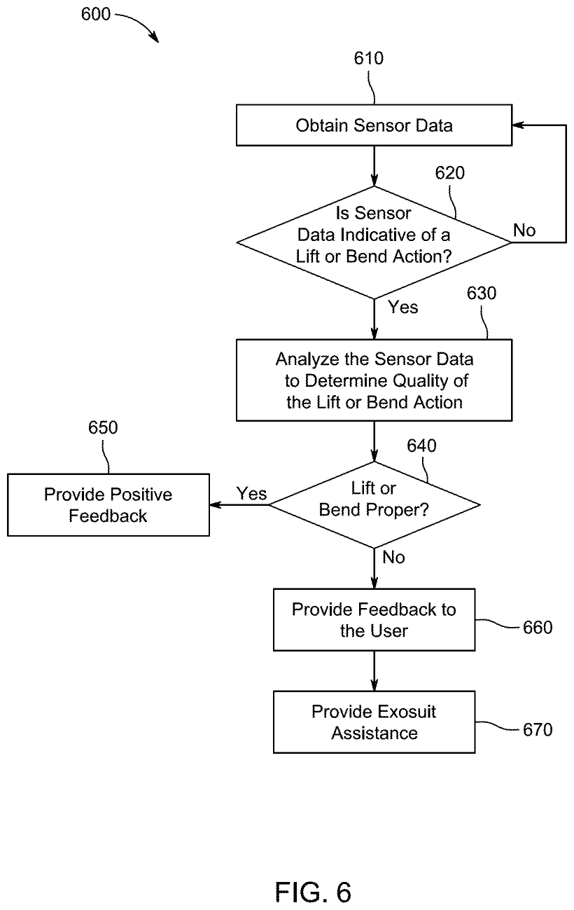

FIG. 6 shows illustrative process 600 for detecting and analyzing bends and lifts according to an embodiment. Process 600 may begin at step 610 by obtaining sensor data. Depending on the sensor configuration, data may be obtained from one or more sensors. The sensors may include 6-axis IMUs or 3-axis IMUs. The sensor configuration may dictate which algorithms are used to analyze the data for proper lift and bend events. At step 620, a determination is made as to whether the sensor data is indicative of a lift or bend action. In step 620, a processor can perform a preliminary analysis of the data to determine whether a participant is engage in a bend or lift action. This preliminary analysis can filter out other actions such as walking, standing, running, etc. that may have data signatures that resemble that of a lift or bend action. If the determination is NO at step 620, process 600 can return to step 610. If the determination is YES at step 620, process 600 can analyze the sensor data to determine quality of the lift or bend action (step 630). Based on the analysis of the sensor data, the lift or bend can classified as proper (safe) or not proper (not safe) at step 640. If the lift or bend is proper, process 600 can provide positive feedback, for example, at step 650. If the lift or bend is not proper, corrective feedback may be provided to the user, at step 660. In some embodiments, an exosuit may provide corrective feedback to assist the user in performing a safe lift or bend action (step 670).

It should be understood that the steps shown in FIG. 6 are illustrative and that additional steps may be added.

There are a variety of different approaches that can be used to detect the impulse-like signatures. In one implementation, a finite state machine, with transitions based on acceleration magnitude thresholds and timing threshold, can be used to detect each impulse in the sequence. The thresholds are chosen to be robust to signal noise and allow for a variety of different lifts, including fast and slow lifts. Furthermore, some signal processing techniques, such as low-pass filters, may be also required to aid in rejecting noise. Other approaches to detecting the impulse-like signatures include using statistical methods, such as moving averages, as well as autoregression and autocorrelation methods. More automatic detection can be achieved using machine learning approaches, which include recurrent neural networks, long short-term memory networks, deep learning classifiers.

In addition to accurately detecting true lifting and bending events, the approach must also reject non-lifting events that generate similar sensor readings. For example, the dynamic motion generated by walking can lead to accelerometer readings that have similar impulse-like signatures. In the preferred state machine implementation, specific temporal thresholds are used to reject candidate lifts that occurred too rapidly, as lifting is much slower than the repetitive, high frequency motion of walking.

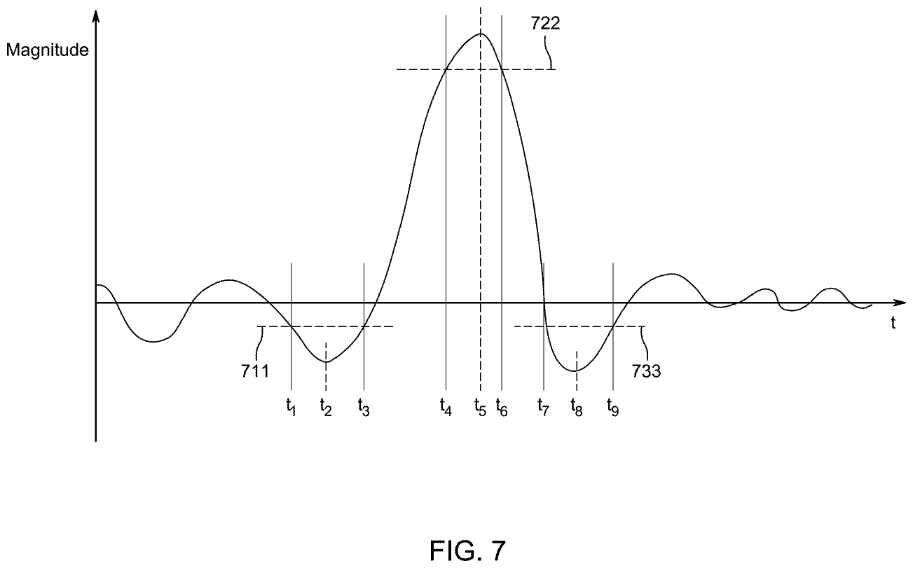

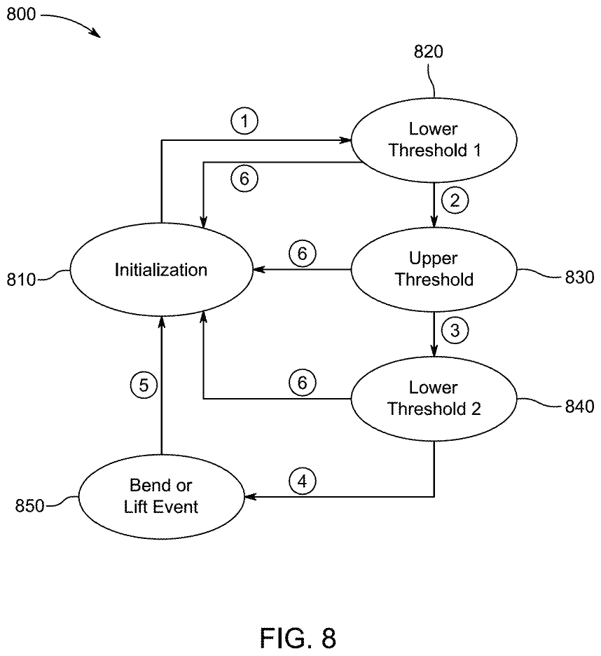

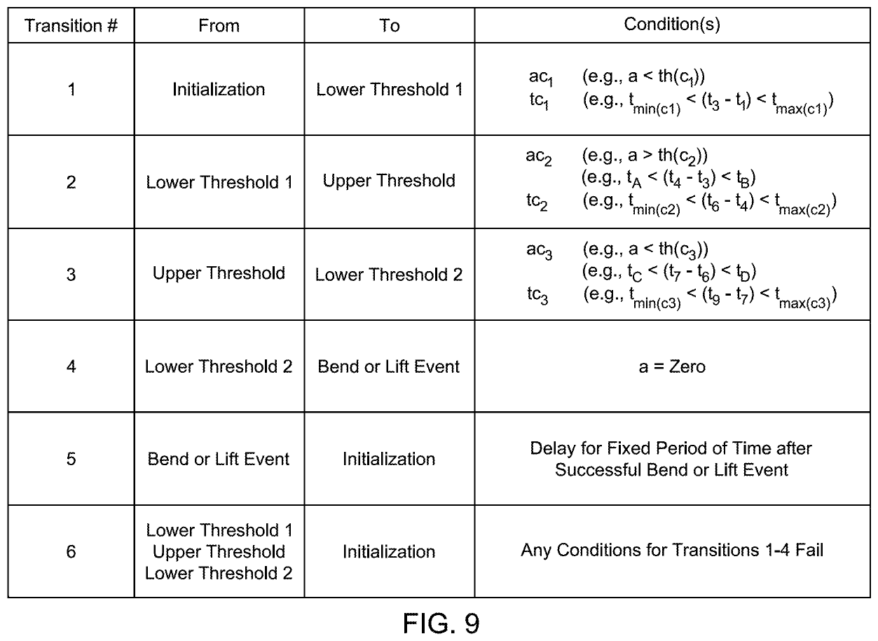

FIG. 7 shows with more specificity an illustrative acceleration timing diagram showing the impulse of an exemplary lift action. The acceleration curve starts at near zero, reaches first low magnitude at time, t2, and then reaches high magnitude at time, t5, and then reaches second low magnitude at time, t8, and then settles back to zero. At times, t1 and t3, the acceleration magnitude crosses threshold_{C1} 711. At times, t4 and t6, the acceleration magnitude crosses threshold_{C2} 722. At times, t7 and t9, the acceleration magnitude crosses threshold_{C3} 733. As will be explained below, the magnitude thresholds, C1-C3, are selected as conditions for a state machine (discussed in connection with FIG. 8). Moreover, the timing of time points t1-t9 are also subject to conditions for the state machine. These conditions are discussed below in connection with FIGS. 8 and 9.

FIG. 8 shows illustrative state machine 800 for determining whether a lift or bend event has occurred when the sensor data is based on accelerometer only data. State machine 800 can include initialization state 810, lower threshold 1 state 820, upper threshold state 830, lower threshold 2 state 840, and bend or lift event state 850. State machine can progress from one state to another based on satisfaction of conditions set forth in conditions table shown in FIG. 9. State machine 800 may begin at initialization state 810. In initialization state 810, a control system may have calibrated the one or more sensors and is ready to monitor sensor data to determine whether the data satisfies requirements of a bend or lift action. In some embodiments, the initialization state may begin when the accelerometer magnitude is at steady state or zero movement. State machine 800 has to satisfy conditions to transition through states 820, 830, and 840 to arrive at bend or lift event state 850. If the conditions fail at any point during the progression from state 810 to state 850, process 800 may revert back to state 810. The conditions set forth in FIG. 9 can be customized for a particular user or updated based on heuristics or at regular intervals.

State machine 800 may perform transition #1 (from state 810 to state 820) when acceleration magnitude condition 1 (AC1) and time condition 1 (TC1) are satisfied. For example, AC1 may refer to the condition of when the acceleration magnitude crosses threshold_{C1} 711 (e.g., A<th_{(C1)}. TC1 may refer to the time period condition for when the acceleration magnitude exist below threshold_{C1} 711. For example, if t_{min(C1)}<(t3−t1)<t_{max(C1)}, the TC1 condition may be satisfied.

State machine 800 may perform transition #2 (from state 820 to state 830) when acceleration magnitude condition 2 (AC2) and time condition 2 (TC2) are satisfied. For example, AC2 may refer to the condition of when the acceleration magnitude crosses threshold_{C2} 722 (e.g., A>th_{(C2)}. TC2 may include two conditions. A first time condition may refer to time period between t3 and t4. For example, when tA<(t4−t3)<tB, then the condition may be satisfied. A second time condition may refer to the time period condition for when the acceleration magnitude exists above threshold_{C2} 722. For example, if t_{min(C2)}<(t7−t6)<t_{max(C2)}, the TC2 condition may be satisfied.

State machine 800 may perform transition #3 (from state 830 to state 840) when acceleration magnitude condition 3 (AC3) and time condition 3 (TC3) are satisfied. For example, AC3 may refer to the condition of when the acceleration magnitude crosses threshold_{C3} 733 (e.g., A<th_{(C3)}. TC3 may include two conditions. A first time condition may refer to time period between t6 and t7. For example, when tC<(t7−t6)<tD, then the condition may be satisfied. A second time condition may refer to the time period condition for when the acceleration magnitude exists below threshold_{C3} 733. For example, if t_{min(C3)}<(t9−t7)<t_{max(C3)}, the TC2 condition may be satisfied.

State machine 800 may perform transition #4 (from state 840 to state 850) when the acceleration returns to zero or steady state. When this condition is satisfied, and state machine 800 transitions to state 850, the conditions for a detected lift or bend action are satisfied and a lift or bend action is present. State machine 800 may perform transition #5 (from state 850 to 810) after successful completion of the lift or bend event or after expiration of a fixed period of time after reaching state 850. State machine 800 perform transition #6 (from state 820, 830, or 840 to state 810) when any of conditions for transitions 1-4 fail.

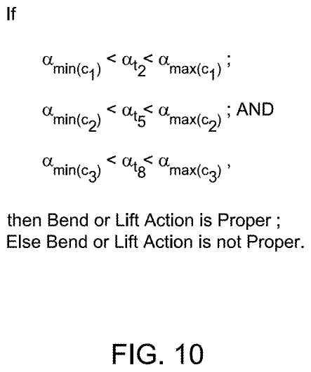

When state machine 800 confirms that the data is indicative of a lift or bend event, further analysis can be performed to determine the quality of the lift or bend event. In one approach, the analysis can examine the pitch angles at or near the first low magnitude (e.g., any one or more acceleration magnitudes between times, t1 and t3), the high magnitude (e.g., any one or more acceleration magnitudes between times t4 and t6), and the second low magnitude (e.g., any one or more acceleration magnitudes between times t7 and t9). In some embodiments the pitch angles at the respective peak magnitudes for the first and second low magnitudes (at times t2 and t8) and the high magnitude (at time t5) may be compared to reference pitch angles for each of the first and second low magnitudes and high magnitude. If the pitch angles fall within a predetermined range for each of the first and second low magnitudes and high magnitude, the bend or lift action may be classified as safe or proper. FIG. 10 shows illustrative pseudocode for analyzing the quality of the lift or bend according to an embodiment. If desired, the analysis step may be implemented as a sub-state machine within state 850 or it can be implemented as a separate process from state machine 800.

When only the accelerometer data is available, the quality of lift can be determined by measuring the change of angles between the states defined by the impulse-like signatures. The impulse-like signatures indicate the progression of the lifting cycle by detecting whether the user is descending or rising. The angle of the upper body can be computed via trigonometry by using the calibrated acceleration components. By comparing the angles when the user is at the bottom of the lift compared to upright position, the amount of bend during the lift can be readily quantified. The bending angle signals might require signal processing techniques, such as low-pass filters, to reduce the effect of noise. In one example, the quality of a lift may be determined by the angle change. Relatively large angle change corresponds to excessive bending, which is indicative of a lower quality lift. Conversely, a relatively small angle change corresponds to less bending, which is indicative of a higher quality lift. The angles, sometimes referred to herein as pitch angles, can be derived from the acceleration readings provided by the accelerometer.

When both accelerometers and gyroscopes are available, a second implementation that separates measurements into a reference frame that is centered around the human user via sensor fusion can be a method to detect an event. Sensor fusion, which combines kinematic data from various sensors to reduce uncertainty, can be used to estimate the orientation with respect to gravity and determine the motion with respect to the human body. When the accelerations can be expressed as vertical and forward components, candidate segmentation markers can be used when the component accelerations surpass specific thresholds. The accelerations within these candidate markers can be then integrated to obtain displacements, which are subsequently used to validate the event. For example, a candidate is only deemed a lifting or bending event if there is a vertical displacement of 0.5 m downwards, and returns to within 0.1 m of the original height. Furthermore, a bending event can be detected by relatively short vertical displacements and relatively large angle changes compared to relatively large vertical displacements and relatively low changes in angle posture. Moreover, use of gyroscopes enables sensor fusion algorithms to be used to provide more precise calculation of pitch angles (compared to pitch angles derived solely from accelerometer data).

The addition of the magnetometer sensor to the accelerometer and gyroscope set allows for more consistent measurements of orientation, in particular with respect to transverse plane. Although lifting is typically focused on the sagittal plane, less error in the transverse plane allows for greater accuracy in determining the forward component, which is important in evaluating the quality of the biomechanic form of the lift.

In addition, the gyroscope and/or magnetometer can be used to measure the change in angular acceleration, velocity and overall rotation in the transverse plane. This can be used to measure the amount of twisting rotation that occurs in each lifting event, including the total rotation displacement, peak angular velocity and peak angular accelerations. Significant changes in rotations can also be used to detect independent twisting events if a lifting event was not detected.

When lifting and bending events have been detected, the time-series data can be analyzed to determine the quality of the biomechanic form. Bending at the back and lifting with straight legs puts strain on the lower back and is often a precursor to injury, while engaging core muscles and using leg muscles to lift provides significantly more power and stability. There are many approaches to determining which lifting event has occurred.

In other implementations where sensor fusion is applicable, the bending angle can be computed in a more robust fashion that is independent of the speed of the lift throughout the entire event. Furthermore, the lifting trajectory can be measured directly and used to determine the quality of the lift. For example, lifts with a forward displacement of greater than 0.4 m indicate bending with the back and poor biomechanic form. In addition, bending angles are more accurate and can be used to help characterize a good versus bad lift event.

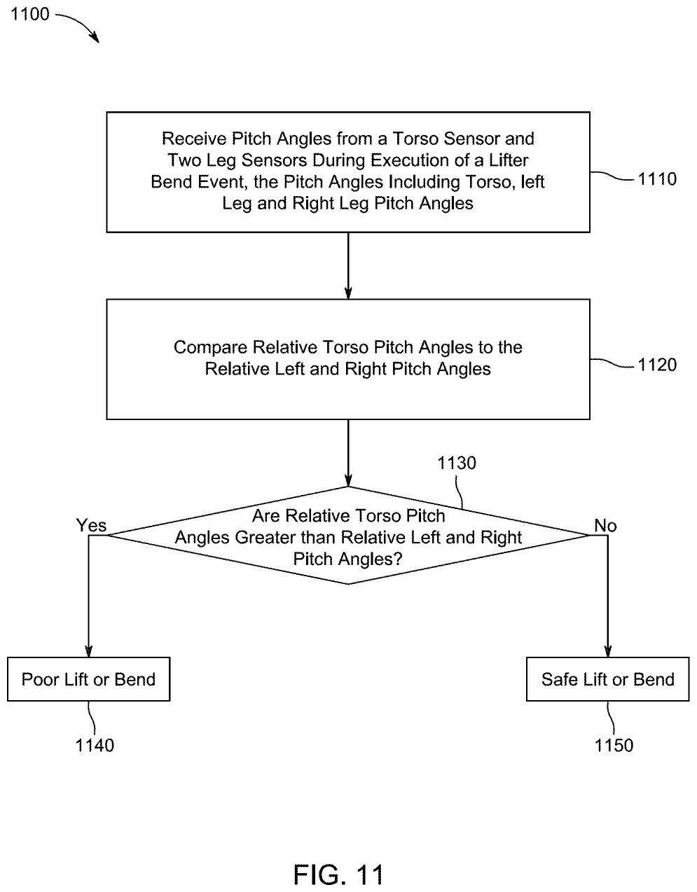

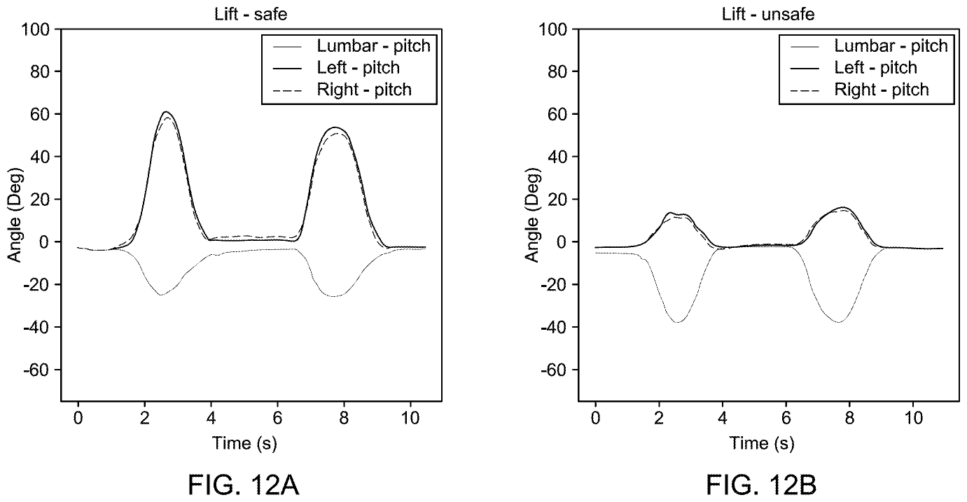

FIG. 11 shows an illustrative process 1100 for evaluating pitch angles from multiple sensors according to an embodiment. Starting at step 1110, pitch angles can be received from a torso sensor and two leg sensor during execution of a lift or bend event. The pitch angles can include torso (e.g., lumbar), left leg, and right leg pitch angles. For example, the torso and leg sensors can be 6-axis IMUs that integrated into an exosuit. FIGS. 12A and 12B show pitch angles obtained from lumbar, left, and right sensors during lifts considered unsafe (i.e., FIG. 12A) and safe (FIG. 12B). At step 1120, the relative torso pitch angles are compared to the relative left and right pitch angles. That is, for a given time stamp, the torso pitch angle is compared to the left and right pitch angles. At step 1130, if the relative torso pitch angles are greater than the relative left and right pitch angles, then the lift or bend is classified as a poor, but if relative torso pitch angles are less than the relative left and right pitch angles, the lift or bend is classified as safe. The degree to which torso pitch angles need differ from the leg pitch angles in order to be classified as a safe or unsafe lift may vary. In some embodiments, the differential may need to exceed to a first threshold, or the differential may merely be absolute.

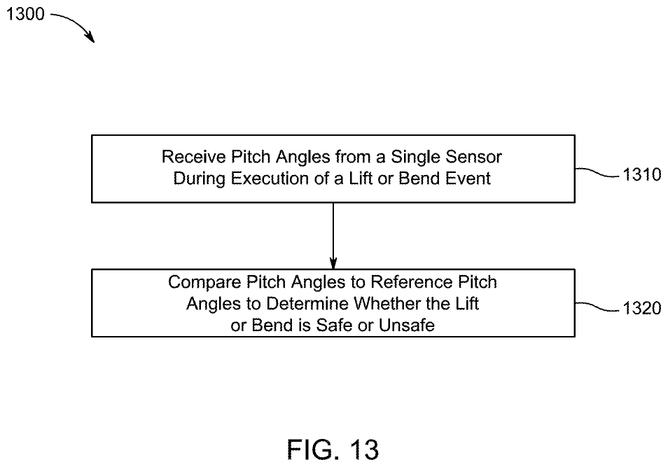

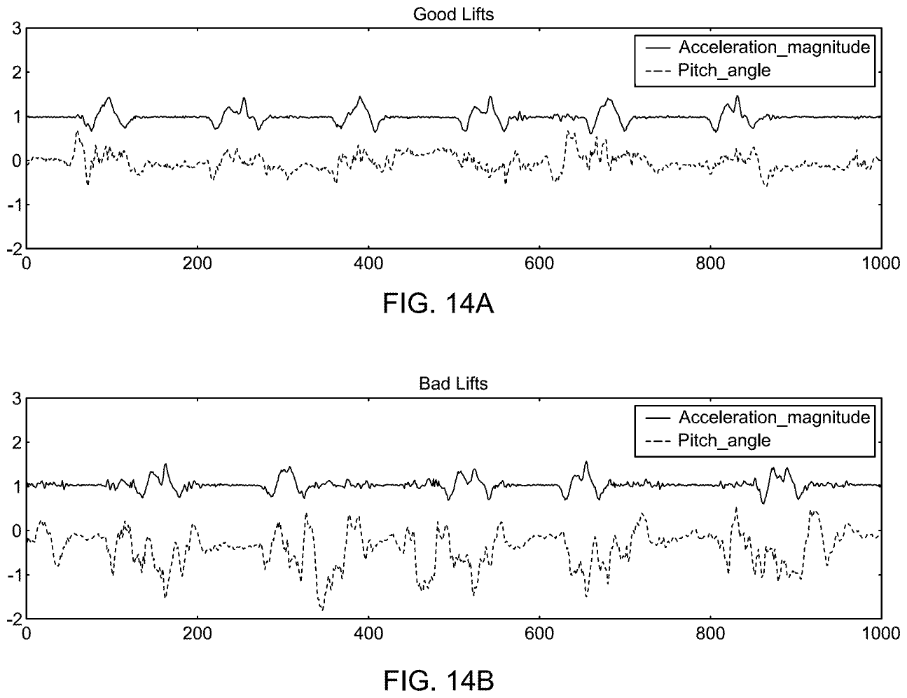

FIG. 13 shows an illustrative process 1300 for determining quality of a lift or bend based on only one 6-axis sensor. For example, a single 6-axis IMU sensor may be used and placed on the torso region of the exosuit. Starting with step 1310, pitch angles can be received from a single sensor during execution of a lift or bend event. At step 1320, the received pitch angles are compared to reference pitch angles or thresholds to determine whether the lift or bend is safe or unsafe. For example, referring to FIGS. 14A and 14B, which show good and bad lifts, respectively, and also show acceleration magnitude and pitch angle. The data shown is derived from one sensor. The impulse signatures represent lift events, and the pitch angles received during the lift events are analyzed. In FIG. 14A, the pitch angles are relatively modest in displacement compared to the pitch angles in FIG. 14B. In one approach, the comparison can determine whether a mean or median of the pitch angel displacement exceeds a threshold to asses whether the lift is safe or not.

In one implementation of process 1300, when only one sensor device is available and located on the pelvis region, a bending event can be detected by the significant changes in angle from the pelvis (in the sagittal plane) and relatively low changes in vertical displacement of the pelvis. Lifting events can be detected by meeting specific vertical displacement thresholds such as 0.5 m, and quality of the lift can be determined by peak acceleration and average speed of lifting event, and maximum angle changes during lifting event.

In addition, peak angular accelerations and velocities in the transverse plane can be used to measure the amount of twisting rotation during a lifting event. Thresholds such as peak angular acceleration, peak angular velocity, and overall rotation can be used to quantify the quality of a lifting event. Lifting events with low amounts of transverse motion is preferable to lifting events with large amounts of transverse motion. For example, a lifting event with angular acceleration greater than 100 degrees/second/second or velocities of greater than 50 degrees/second can be categorized as a poor lifting event. Generally, lifting events with higher peak angular accelerations and velocities increase the risk of injury and are a sign of poor control throughout a lifting event.

After a lifting or bending event is detected and analyzed, real-time feedback can be provided to the user. For example, real-time feedback can be provided with haptic vibration via the exosuit. In some embodiments, the exosuit may provide assistance to compensate for improper form. In addition, feedback can also be provided via audio through a wireless audio earpiece, headphones, or audio speaker that is integrated in the exosuit. In addition, auditory beeps, chimes or jingles can be used to provide the feedback. Text messages, notification popups, and email summaries can also be used to provide feedback to a user, manager, and/or a team on their progress, tips they should focus on, and other relevant feedback.

Sensor data can be synchronized with a client software application running on a smart phone, computer or other computing peripheral device. The client application can be used to track a user's progress, count the number of lifting and bending events, and score each event. Events can be displayed on a user interface and marked with an indicator for good, bad, or OK. User's can configure which events to track and receive coaching on. For example, in an industrial application, the user can configure the device to provide only reminders and coaching feedback for Lifting events. A different user may configure the device to vibrate whenever a bending event is detected as a way to help remind and coach the user to reduce the number of bending events. For warehouse workers, proper lifting technique is important for worker productivity and health. Improper lifting events and bends can greatly increase the worker's risk of injury and effect a company's bottom line from productivity loss to worker's compensation. The data from each user and sensor can be synchronized with a centralized application or database that can enable and aggregate the monitoring of groups of workers or the entire workplace operations.

Over time, the sensor and system can monitor the quality of each individual lifting event during a workday, week, or month. Each lifting event can be compared against a user's individual baseline of lifting parameters. Each lifting event can be compared to the baseline and used to help identify a potential fatigue state. Also indicators can be averaged over a number of recent lifting events which can then be compared to the baseline. For example, the variance of the previous 5 lifting event metrics in an hour can be compared to a baseline average. If the variance increases significantly across the five lifting events, relative to the baseline variance, then the sensor and system may alert the user to be mindful of potential fatigue, or flag for review to the company.

Biomechanical indicators that can be used to measure and predict fatigue may include peak angular and displacement accelerations or velocities, pelvis stability, and variance between lifting events. Stability can be determined by significant changes in linear or angular accelerations and velocities, range of motion, and the variance of these metrics over time. For example, someone that is not fatigued may demonstrate consistent lifting behaviors with low variances across lifting events. Someone that may be experiencing fatigue may exhibit lifting events that have high variance across lifting events. Specific metrics such as peak angular rotation speed, displacement speed and impulse acceleration may vary significantly between lifting events hinting that the user may not be able to maintain control throughout the lifting event. In other instances, lifting parameters may exceed specific thresholds that are absolute in value or relative, such as to an individual's baseline.

The sensor and system can be used to monitor a group of workers in an industrial environment and track the productivity and risk across the group of workers. If an individual worker's biomechanics of lifting begin to degrade, an alert can be sent to the worker to be mindful of their lifting behaviors. In addition, the system can dynamically alert the management or direct the user to take a break and identify a different worker to take the previous worker's place.

Data from the sensors and system can be synchronized with a cloud database that aggregates all user data across the entire working group or user base. Lifting and bending quality data can be overlaid with GPS coordinates to identify a heatmap of locations on the industrial operation's floor where (or when) most poor lifting events are detected. A dashboard of high risk individuals, locations, or times can be identified and shared with management.

As management makes changes to a worker's schedule, training, or environment, new resulting biomechanical data is analyzed and stored. This longitudinal data can be used to build prediction models to help a management team inform on their priorities and decision making. Predictive models can be developed to identify optimal working operations that balance productivity and injury risk.

In addition, predictive models can be built to detect when a user is beginning to fatigue or which workers are most susceptible to injury. Data can be used to also identify which operations, locations, times, and particular tasks lead to the most injuries, and help management identify weaknesses within their operations and safety protocols. In addition, some functions may be identified for automation by the system and help the management team reallocate workers to higher value tasks and activities. This can help bring down the overall injury risk profile while increasing overall productivity.

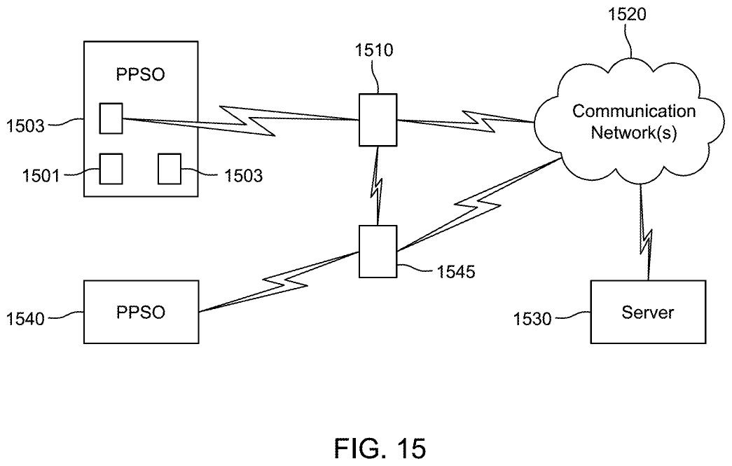

FIG. 15 illustrates an example exosuit 1500 that includes actuators 1501, sensors 1503, and a controller configured to operate elements of exosuit 1500 (e.g., 1501, 1503) to enable functions of the exosuit 1500. The controller 1505 is configured to communicate wirelessly with a user interface 1510. The user interface 1510 is configured to present information to a user (e.g., a wearer of the exosuit 1500) and to the controller 1505 of the flexible exosuit or to other systems. The user interface 1510 can be involved in controlling and/or accessing information from elements of the exosuit 1500. For example, an application being executed by the user interface 1510 can access data from the sensors 1503, calculate an operation (e.g., to apply dorsiflexion stretch) of the actuators 1501, and transmit the calculated operation to the exosuit 1500. The user interface 1510 can additionally be configured to enable other functions; for example, the user interface 1510 can be configured to be used as a cellular telephone, a portable computer, an entertainment device, or to operate according to other applications.

The user interface 1510 can be configured to be removably mounted to the exosuit 1500 (e.g., by straps, magnets, Velcro, charging and/or data cables). Alternatively, the user interface 1510 can be configured as a part of the exosuit 1500 and not to be removed during normal operation. In some examples, a user interface can be incorporated as part of the exosuit 1500 (e.g., a touchscreen integrated into a sleeve of the exosuit 1500) and can be used to control and/or access information about the exosuit 1500 in addition to using the user interface 1510 to control and/or access information about the exosuit 1500. In some examples, the controller 1505 or other elements of the exosuit 1500 are configured to enable wireless or wired communication according to a standard protocol (e.g., Bluetooth, ZigBee, WiFi, LTE or other cellular standards, IRdA, Ethernet) such that a variety of systems and devices can be made to operate as the user interface 1510 when configured with complementary communications elements and computer-readable programs to enable such functionality.

The exosuit 1500 can be configured as described in example embodiments herein or in other ways according to an application. The exosuit 1500 can be operated to enable a variety of applications. The exosuit 1500 can be operated to enhance the strength of a wearer by detecting motions of the wearer (e.g., using sensors 1503) and responsively applying torques and/or forces to the body of the wearer (e.g., using actuators 1501) to increase the forces the wearer is able to apply to his/her body and/or environment. The exosuit 1500 can be operated to train a wearer to perform certain physical activities. For example, the exosuit 1500 can be operated to enable rehabilitative therapy of a wearer. The exosuit 1500 can operate to amplify motions and/or forces produced by a wearer undergoing therapy in order to enable the wearer to successfully complete a program of rehabilitative therapy. Additionally or alternatively, the exosuit 1500 can be operated to prohibit disordered movements of the wearer and/or to use the actuators 1501 and/or other elements (e.g., haptic feedback elements) to indicate to the wearer a motion or action to perform and/or motions or actions that should not be performed or that should be terminated. Similarly, other programs of physical training (e.g., dancing, skating, other athletic activities, vocational training) can be enabled by operation of the exosuit 1500 to detect motions, torques, or forces generated by a wearer and/or to apply forces, torques, or other haptic feedback to the wearer. Other applications of the exosuit 1500 and/or user interface 1510 are anticipated.

The user interface 1510 can additionally communicate with communications network(s) 1520. For example, the user interface 1510 can include a WiFi radio, an LTE transceiver or other cellular communications equipment, a wired modem, or some other elements to enable the user interface 1510 and exosuit 1500 to communicate with the Internet. The user interface 1510 can communicate through the communications network 1520 with a server 1530. Communication with the server 1530 can enable functions of the user interface 1510 and exosuit 1500. In some examples, the user interface 1510 can upload telemetry data (e.g., location, configuration of elements 1501, 1503 of the exosuit 1500, physiological data about a wearer of the exosuit 1500) to the server 1530.

In some examples, the server 1530 can be configured to control and/or access information from elements of the exosuit 1500 (e.g., 1501, 1503) to enable some application of the exosuit 1500. For example, the server 1530 can operate elements of the exosuit 1500 to move a wearer out of a dangerous situation if the wearer was injured, unconscious, or otherwise unable to move themselves and/or operate the exosuit 1500 and user interface 1510 to move themselves out of the dangerous situation. Other applications of a server in communications with a exosuit are anticipated.

The user interface 1510 can be configured to communicate with a second user interface 1545 in communication with and configured to operate a second flexible exosuit 1540. Such communication can be direct (e.g., using radio transceivers or other elements to transmit and receive information over a direct wireless or wired link between the user interface 1510 and the second user interface 1545). Additionally or alternatively, communication between the user interface 1510 and the second user interface 1545 can be facilitated by communications network(s) 1520 and/or a server 1530 configured to communicate with the user interface 1510 and the second user interface 1545 through the communications network(s) 1520.

Communication between the user interface 1510 and the second user interface 1545 can enable applications of the exosuit 1500 and second exosuit 1540. In some examples, actions of the exosuit 1500 and second flexible exosuit 1540 and/or of wearers of the exosuit 1500 and second exosuit 1540 can be coordinated. For example, the exosuit 1500 and second exosuit 1540 can be operated to coordinate the lifting of a heavy object by the wearers. The timing of the lift, and the degree of support provided by each of the wearers and/or the exosuit 1500 and second exosuit 1540 can be controlled to increase the stability with which the heavy object was carried, to reduce the risk of injury of the wearers, or according to some other consideration. Coordination of actions of the exosuit 1500 and second exosuit 1540 and/or of wearers thereof can include applying coordinated (in time, amplitude, or other properties) forces and/or torques to the wearers and/or elements of the environment of the wearers and/or applying haptic feedback (though actuators of the exosuits 1500, 1540, through dedicated haptic feedback elements, or through other methods) to the wearers to guide the wearers toward acting in a coordinated manner.

Coordinated operation of the exosuit 1500 and second exosuit 1540 can be implemented in a variety of ways. In some examples, one exosuit (and the wearer thereof) can act as a master, providing commands or other information to the other exosuit such that operations of the exosuit 1500, 1540 are coordinated. For example, the exosuit 1500, 1540 can be operated to enable the wearers to dance (or to engage in some other athletic activity) in a coordinated manner. One of the exosuits can act as the ‘lead’, transmitting timing or other information about the actions performed by the ‘lead’ wearer to the other exosuit, enabling coordinated dancing motions to be executed by the other wearer. In some examples, a first wearer of a first exosuit can act as a trainer, modeling motions or other physical activities that a second wearer of a second exosuit can learn to perform. The first exosuit can detect motions, torques, forces, or other physical activities executed by the first wearer and can send information related to the detected activities to the second exosuit. The second exosuit can then apply forces, torques, haptic feedback, or other information to the body of the second wearer to enable the second wearer to learn the motions or other physical activities modeled by the first wearer. In some examples, the server 1530 can send commands or other information to the exosuits 1500, 1540 to enable coordinated operation of the exosuits 1500, 1540.

The exosuit 1500 can be operated to transmit and/or record information about the actions of a wearer, the environment of the wearer, or other information about a wearer of the exosuit 1500. In some examples, kinematics related to motions and actions of the wearer can be recorded and/or sent to the server 1530. These data can be collected for medical, scientific, entertainment, social media, or other applications. The data can be used to operate a system. For example, the exosuit 1500 can be configured to transmit motions, forces, and/or torques generated by a user to a robotic system (e.g., a robotic arm, leg, torso, humanoid body, or some other robotic system) and the robotic system can be configured to mimic the activity of the wearer and/or to map the activity of the wearer into motions, forces, or torques of elements of the robotic system. In another example, the data can be used to operate a virtual avatar of the wearer, such that the motions of the avatar mirrored or were somehow related to the motions of the wearer. The virtual avatar can be instantiated in a virtual environment, presented to an individual or system with which the wearer is communicating, or configured and operated according to some other application.

Conversely, the exosuit 1500 can be operated to present haptic or other data to the wearer. In some examples, the actuators 1501 (e.g., twisted string actuators, exotendons) and/or haptic feedback elements (e.g., EPAM haptic elements) can be operated to apply and/or modulate forces applied to the body of the wearer to indicate mechanical or other information to the wearer. For example, the activation in a certain pattern of a haptic element of the exosuit 1500 disposed in a certain location of the exosuit 1500 can indicate that the wearer had received a call, email, or other communications. In another example, a robotic system can be operated using motions, forces, and/or torques generated by the wearer and transmitted to the robotic system by the exosuit 1500. Forces, moments, and other aspects of the environment and operation of the robotic system can be transmitted to the exosuit 1500 and presented (using actuators 1580 or other haptic feedback elements) to the wearer to enable the wearer to experience force-feedback or other haptic sensations related to the wearer's operation of the robotic system. In another example, haptic data presented to a wearer can be generated by a virtual environment, e.g., an environment containing an avatar of the wearer that is being operated based on motions or other data related to the wearer that is being detected by the exosuit 1500.

Note that the exosuit 1500 illustrated in FIG. 15 is only one example of an exosuit that can be operated by control electronics, software, or algorithms described herein. Control electronics, software, or algorithms as described herein can be configured to control flexible exosuits or other mechatronic and/or robotic system having more, fewer, or different actuators, sensors or other elements. Further, control electronics, software, or algorithms as described herein can be configured to control exosuits configured similarly to or differently from the illustrated exosuit 1500. Further, control electronics, software, or algorithms as described herein can be configured to control flexible exosuits having reconfigurable hardware (i.e., exosuits that are able to have actuators, sensors, or other elements added or removed) and/or to detect a current hardware configuration of the flexible exosuits using a variety of methods.

A controller of a exosuit and/or computer-readable programs executed by the controller can be configured to provide encapsulation of functions and/or components of the flexible exosuit. That is, some elements of the controller (e.g., subroutines, drivers, services, daemons, functions) can be configured to operate specific elements of the exosuit (e.g., a twisted string actuator, a haptic feedback element) and to allow other elements of the controller (e.g., other programs) to operate the specific elements and/or to provide abstracted access to the specific elements (e.g., to translate a command to orient an actuator in a commanded direction into a set of commands sufficient to orient the actuator in the commanded direction). This encapsulation can allow a variety of services, drivers, daemons, or other computer-readable programs to be developed for a variety of applications of a flexible exosuits. Further, by providing encapsulation of functions of a flexible exosuit in a generic, accessible manner (e.g., by specifying and implementing an application programming interface (API) or other interface standard), computer-readable programs can be created to interface with the generic, encapsulated functions such that the computer-readable programs can enable operating modes or functions for a variety of differently-configured exosuit, rather than for a single type or model of flexible exosuit. For example, a virtual avatar communications program can access information about the posture of a wearer of a flexible exosuit by accessing a standard exosuit API. Differently-configured exosuits can include different sensors, actuators, and other elements, but can provide posture information in the same format according to the API. Other functions and features of a flexible exosuit, or other robotic, exoskeletal, assistive, haptic, or other mechatronic system, can be encapsulated by APIs or according to some other standardized computer access and control interface scheme.

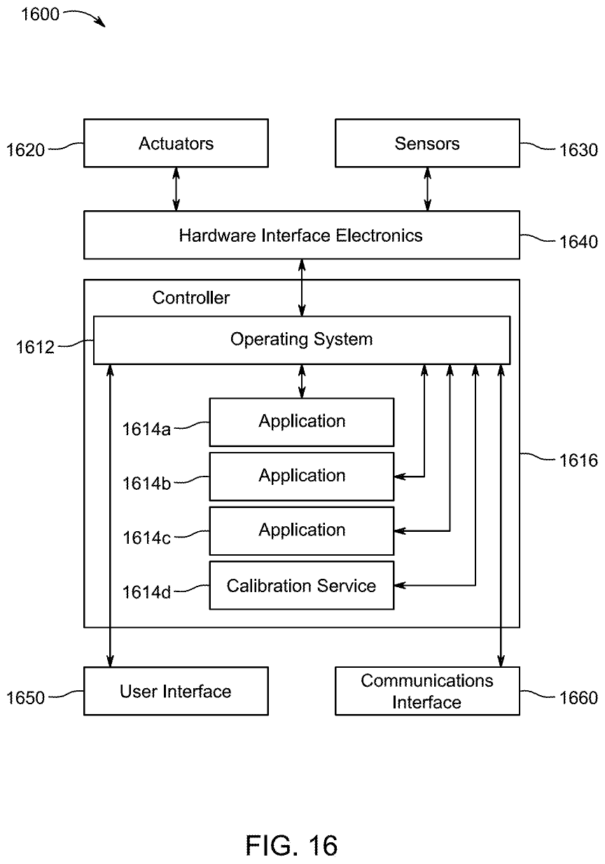

FIG. 16 is a schematic illustrating elements of a exosuit 1600 and a hierarchy of control or operating the exosuit 1600. The flexible exosuit includes actuators 1620 and sensors 1630 configured to apply forces and/or torques to and detect one or more properties of, respectively, the exosuit 1600, a wearer of the exosuit 1600, and/or the environment of the wearer. The exosuit 1600 additionally includes a controller 1610 configured to operate the actuators 1620 and sensors 1630 by using hardware interface electronics 1640. The hardware electronics interface 1640 includes electronics configured to interface signals from and to the controller 1610 with signals used to operate the actuators 1620 and sensors 1630. For example, the actuators 1620 can include exotendons, and the hardware interface electronics 1640 can include high-voltage generators, high-voltage switches, and high-voltage capacitance meters to clutch and un-clutch the exotendons and to report the length of the exotendons. The hardware interface electronics 1640 can include voltage regulators, high voltage generators, amplifiers, current detectors, encoders, magnetometers, switches, controlled-current sources, DACs, ADCs, feedback controllers, brushless motor controllers, or other electronic and mechatronic elements.

The controller 1610 additionally operates a user interface 1650 that is configured to present information to a user and/or wearer of the exosuit 1600 and a communications interface 1960 that is configured to facilitate the transfer of information between the controller 1610 and some other system (e.g., by transmitting a wireless signal). Additionally or alternatively, the user interface 1650 can be part of a separate system that is configured to transmit and receive user interface information to/from the controller 1610 using the communications interface 1960 (e.g., the user interface 1650 can be part of a cellphone).

The controller 1610 is configured to execute computer-readable programs describing functions of the flexible exosuit 1612. Among the computer-readable programs executed by the controller 1610 are an operating system 1612, applications 1614 a, 1614 b, 1614 c, and a calibration service 1616. The operating system 1612 manages hardware resources of the controller 1610 (e.g., I/O ports, registers, timers, interrupts, peripherals, memory management units, serial and/or parallel communications units) and, by extension, manages the hardware resources of the exosuit 1600. The operating system 1612 is the only computer-readable program executed by the controller 1610 that has direct access to the hardware interface electronics 1640 and, by extension, the actuators 1620 and sensors 1630 of the exosuit 1600.

The applications 1614 a, 1614 b, 1614 are computer-readable programs that describe some function, functions, operating mode, or operating modes of the exosuit 1600. For example, application 1614 a can describe a process for transmitting information about the wearer's posture to update a virtual avatar of the wearer that includes accessing information on a wearer's posture from the operating system 1612, maintaining communications with a remote system using the communications interface 1660, formatting the posture information, and sending the posture information to the remote system. The calibration service 1616 is a computer-readable program describing processes to store parameters describing properties of wearers, actuators 1620, and/or sensors 1630 of the exosuit 1600, to update those parameters based on operation of the actuators 1620, and/or sensors 1630 when a wearer is using the exosuit 1600, to make the parameters available to the operating system 1612 and/or applications 1614 a, 1614 b, 1614 c, and other functions relating to the parameters. Note that applications 1614 a, 1614 b, 1614 and calibration service 1616 are intended as examples of computer-readable programs that can be run by the operating system 1612 of the controller 1610 to enable functions or operating modes of a exosuit 1600.

The operating system 1612 can provide for low-level control and maintenance of the hardware (e.g., 1620, 1630, 1640). In some examples, the operating system 1612 and/or hardware interface electronics 1640 can detect information about the exosuit 1600, the wearer, and/or the wearer's environment from one or more sensors 1630 at a constant specified rate. The operating system 1612 can generate an estimate of one or more states or properties of the exosuit 1600 or components thereof using the detected information. The operating system 1612 can update the generated estimate at the same rate as the constant specified rate or at a lower rate. The generated estimate can be generated from the detected information using a filter to remove noise, generate an estimate of an indirectly-detected property, or according to some other application. For example, the operating system 1612 can generate the estimate from the detected information using a Kalman filter to remove noise and to generate an estimate of a single directly or indirectly measured property of the exosuit 1600, the wearer, and/or the wearer's environment using more than one sensor. In some examples, the operating system can determine information about the wearer and/or exosuit 1600 based on detected information from multiple points in time. For example, the operating system 1600 can determine an eversion stretch and dorsiflexion stretch.

In some examples, the operating system 1612 and/or hardware interface electronics 1640 can operate and/or provide services related to operation of the actuators 1620. That is, in case where operation of the actuators 1620 requires the generation of control signals over a period of time, knowledge about a state or states of the actuators 1620, or other considerations, the operating system 1612 and/or hardware interface electronics 1640 can translate simple commands to operate the actuators 1620 (e.g., a command to generate a specified level of force using a twisted string actuator (TSA) of the actuators 1620) into the complex and/or state-based commands to the hardware interface electronics 1640 and/or actuators 1620 necessary to effect the simple command (e.g., a sequence of currents applied to windings of a motor of a TSA, based on a starting position of a rotor determined and stored by the operating system 1610, a relative position of the motor detected using an encoder, and a force generated by the TSA detected using a load cell).

In some examples, the operating system 1612 can further encapsulate the operation of the exosuit 1600 by translating a system-level simple command (e.g., a commanded level of force tension applied to the footplate) into commands for multiple actuators, according to the configuration of the exosuit 1600. This encapsulation can enable the creation of general-purpose applications that can effect a function of an exosuit (e.g., allowing a wearer of the exosuit to stretch his foot) without being configured to operate a specific model or type of exosuit (e.g., by being configured to generate a simple force production profile that the operating system 1612 and hardware interface electronics 1640 can translate into actuator commands sufficient to cause the actuators 1620 to apply the commanded force production profile to the footplate).

The operating system 1612 can act as a standard, multi-purpose platform to enable the use of a variety of exosuits having a variety of different hardware configurations to enable a variety of mechatronic, biomedical, human interface, training, rehabilitative, communications, and other applications. The operating system 1612 can make sensors 1630, actuators 1620, or other elements or functions of the exosuit 1600 available to remote systems in communication with the exosuit 1600 (e.g., using the communications interface 1660) and/or a variety of applications, daemons, services, or other computer-readable programs being executed by operating system 1612. The operating system 1612 can make the actuators, sensors, or other elements or functions available in a standard way (e.g., through an API, communications protocol, or other programmatic interface) such that applications, daemons, services, or other computer-readable programs can be created to be installed on, executed by, and operated to enable functions or operating modes of a variety of flexible exosuits having a variety of different configurations. The API, communications protocol, or other programmatic interface made available by the operating system 1612 can encapsulate, translate, or otherwise abstract the operation of the exosuit 1600 to enable the creation of such computer-readable programs that are able to operate to enable functions of a wide variety of differently-configured flexible exosuits.

Additionally or alternatively, the operating system 1612 can be configured to operate a modular flexible exosuit system (i.e., a flexible exosuit system wherein actuators, sensors, or other elements can be added or subtracted from a flexible exosuit to enable operating modes or functions of the flexible exosuit). In some examples, the operating system 1612 can determine the hardware configuration of the exosuit 1600 dynamically and can adjust the operation of the exosuit 1600 relative to the determined current hardware configuration of the exosuit 1600. This operation can be performed in a way that was ‘invisible’ to computer-readable programs (e.g., 1614 a, 1614 b, 1614 c) accessing the functionality of the exosuit 1600 through a standardized programmatic interface presented by the operating system 1612. For example, the computer-readable program can indicate to the operating system 1612, through the standardized programmatic interface, that a specified level of torque was to be applied to an ankle of a wearer of the exosuit 1600. The operating system 1612 can responsively determine a pattern of operation of the actuators 1620, based on the determined hardware configuration of the exosuit 1600, sufficient to apply the specified level of torque to the ankle of the wearer.

In some examples, the operating system 1612 and/or hardware interface electronics 1640 can operate the actuators 1620 to ensure that the exosuit 1600 does not operate to directly cause the wearer to be injured and/or elements of the exosuit 1600 to be damaged. In some examples, this can include not operating the actuators 1620 to apply forces and/or torques to the body of the wearer that exceeded some maximum threshold. This can be implemented as a watchdog process or some other computer-readable program that can be configured (when executed by the controller 1610) to monitor the forces being applied by the actuators 1620 (e.g., by monitoring commands sent to the actuators 1620 and/or monitoring measurements of forces or other properties detected using the sensors 1630) and to disable and/or change the operation of the actuators 1620 to prevent injury of the wearer. Additionally or alternatively, the hardware interface electronics 1640 can be configured to include circuitry to prevent excessive forces and/or torques from being applied to the wearer (e.g., by channeling to a comparator the output of a load cell that is configured to measure the force generated by a TSA, and configuring the comparator to cut the power to the motor of the TSA when the force exceeded a specified level).

In some examples, operating the actuators 1620 to ensure that the exosuit 1600 does not damage itself can include a watchdog process or circuitry configured to prevent over-current, over-load, over-rotation, or other conditions from occurring that can result in damage to elements of the exosuit 1600. For example, the hardware interface electronics 1640 can include a metal oxide varistor, breaker, shunt diode, or other element configured to limit the voltage and/or current applied to a winding of a motor.

Note that the above functions described as being enabled by the operating system 1612 can additionally or alternatively be implemented by applications 1614 a, 1614 b, 1614 c, services, drivers, daemons, or other computer-readable programs executed by the controller 1600. The applications, drivers, services, daemons, or other computer-readable programs can have special security privileges or other properties to facilitate their use to enable the above functions.

The operating system 1612 can encapsulate the functions of the hardware interface electronics 1640, actuators 1620, and sensors 1630 for use by other computer-readable programs (e.g., applications 1614 a, 1614 b, 1614 c, calibration service 1616), by the user (through the user interface 1650), and/or by some other system (i.e., a system configured to communicate with the controller 1610 through the communications interface 1960). The encapsulation of functions of the exosuit 1600 can take the form of application programming interfaces (APIs), i.e., sets of function calls and procedures that an application running on the controller 1610 can use to access the functionality of elements of the exosuit 1600. In some examples, the operating system 1612 can make available a standard ‘exosuit API’ to applications being executed by the controller 1610. The ‘exosuit API’ can enable applications 1614 a, 1614 b, 1614 c to access functions of the exosuit 1600 without requiring those applications 1614 a, 1614 b, 1614 c to be configured to generate whatever complex, time-dependent signals are necessary to operate elements of the exosuit 1600 (e.g., actuators 1620, sensors 1630).

The ‘exosuit API’ can allow applications 1614 a, 1614 b, 1614 c to send simple commands to the operating system 1612 (e.g., ‘begin storing mechanical energy from the ankle of the wearer when the foot of the wearer contacts the ground’) in such that the operating system 1612 can interpret those commands and generate the command signals to the hardware interface electronics 1640 or other elements of the exosuit 1600 that are sufficient to effect the simple commands generated by the applications 1614 a, 1614 b, 1614 c (e.g., determining whether the foot of the wearer has contacted the ground based on information detected by the sensors 1630, responsively applying high voltage to an exotendon that crosses the user's ankle).

The ‘exosuit API’ can be an industry standard (e.g., an ISO standard), a proprietary standard, an open-source standard, or otherwise made available to individuals that can then produce applications for exosuits. The ‘exosuit API’ can allow applications, drivers, services, daemons, or other computer-readable programs to be created that are able to operate a variety of different types and configurations of exosuits by being configured to interface with the standard ‘exosuit API’ that is implemented by the variety of different types and configurations of exosuits. Additionally or alternatively, the ‘exosuit API’ can provide a standard encapsulation of individual exosuit-specific actuators (i.e., actuators that apply forces to specific body segments, where differently-configured exosuits may not include an actuator that applies forces to the same specific body segments) and can provide a standard interface for accessing information on the configuration of whatever exosuit is providing the ‘exosuit API’. An application or other program that accesses the ‘exosuit API’ can access data about the configuration of the exosuit (e.g., locations and forces between body segments generated by actuators, specifications of actuators, locations and specifications of sensors) and can generate simple commands for individual actuators (e.g., generate a force of 30 newtons for 50 milliseconds) based on a model of the exosuit generated by the application and based on the information on the accessed data about the configuration of the exosuit. Additional or alternate functionality can be encapsulated by an ‘exosuit API’ according to an application.

Applications 1614 a, 1614 b, 1614 c can individually enable all or parts of the functions and operating modes of a flexible exosuit described herein. For example, an application can enable haptic control of a robotic system by transmitting postures, forces, torques, and other information about the activity of a wearer of the exosuit 1600 and by translating received forces and torques from the robotic system into haptic feedback applied to the wearer (i.e., forces and torques applied to the body of the wearer by actuators 1620 and/or haptic feedback elements). In another example, an application can enable a wearer to locomote more efficiently by submitting commands to and receiving data from the operating system 1612 (e.g., through an API) such that actuators 1620 of the exosuit 1600 assist the movement of the user, extract negative work from phases of the wearer's locomotion and inject the stored work to other phases of the wearer's locomotion, or other methods of operating the exosuit 1600. Applications can be installed on the controller 1610 and/or on a computer-readable storage medium included in the exosuit 1600 by a variety of methods. Applications can be installed from a removable computer-readable storage medium or from a system in communication with the controller 1610 through the communications interface 1960. In some examples, the applications can be installed from a web site, a repository of compiled or un-compiled programs on the Internet, an online store (e.g., Google Play, iTunes App Store), or some other source. Further, functions of the applications can be contingent upon the controller 1610 being in continuous or periodic communication with a remote system (e.g., to receive updates, authenticate the application, to provide information about current environmental conditions).

The exosuit 1600 illustrated in FIG. 16 is intended as an illustrative example. Other configurations of flexible exosuits and of operating systems, kernels, applications, drivers, services, daemons, or other computer-readable programs are anticipated. For example, an operating system configured to operate an exosuit can include a real-time operating system component configured to generate low-level commands to operate elements of the exosuit and a non-real-time component to enable less time-sensitive functions, like a clock on a user interface, updating computer-readable programs stored in the exosuit, or other functions. A exosuit can include more than one controller; further, some of those controllers can be configured to execute real-time applications, operating systems, drivers, or other computer-readable programs (e.g., those controllers were configured to have very short interrupt servicing routines, very fast thread switching, or other properties and functions relating to latency-sensitive computations) while other controllers are configured to enable less time-sensitive functions of a flexible exosuit. Additional configurations and operating modes of an exosuit are anticipated. Further, control systems configured as described herein can additionally or alternatively be configured to enable the operation of devices and systems other than exosuit; for example, control systems as described herein can be configured to operate robots, rigid exosuits or exoskeletons, assistive devices, prosthetics, or other mechatronic devices.

Control of actuators of an exosuit can be implemented in a variety of ways according to a variety of control schemes. Generally, one or more hardware and/or software controllers can receive information about the state of the flexible exosuit, a wearer of the exosuit, and/or the environment of the exosuit from sensors disposed on or within the exosuit and/or a remote system in communication with the exosuit. The one or more hardware and/or software controllers can then generate a control output that can be executed by actuators of the exosuit to affect a commanded state of the exosuit and/or to enable some other application. One or more software controllers can be implemented as part of an operating system, kernel, driver, application, service, daemon, or other computer-readable program executed by a processor included in the exosuit.

In some embodiments, a powered assistive exosuit intended primarily for assistive functions can also be adapted to perform exosuit functions. In one embodiment, an assistive exosuit similar to the embodiments described in US Publication No. 2018/0056104, that is used for assistive functions may be adapted to perform exosuit functions. Embodiments of such an assistive exosuit typically include FLAs approximating muscle groups such as hip flexors, gluteal/hip extensors, spinal extensors, or abdominal muscles. In the assistive modes of these exosuits, these FLAs provide assistance for activities such as moving between standing and seated positions, walking, and postural stability. Actuation of specific FLAs within such an exosuit system may also provide stretching assistance. Typically, activation of one or more FLAs approximating a muscle group can stretch the antagonist muscles. For example, activation of one or more FLAs approximating the abdominal muscles might stretch the spinal extensors, or activation of one or more FLAs approximating gluteal/hip extensor muscles can stretch the hip flexors. The exosuit may be adapted to detect when the wearer is ready to initiate a stretch and perform an automated stretching regimen; or the wearer may indicate to the suit to initiate a stretching regimen.

It can be appreciated that assistive exosuits may have multiple applications. Assistive exosuits may be prescribed for medical applications. These may include therapeutic applications, such as assistance with exercise or stretching regimens for rehabilitation, disease mitigation or other therapeutic purposes. Mobility-assistance devices such as wheelchairs, walkers, crutches and scooters are often prescribed for individuals with mobility impairments. Likewise, an assistive exosuit may be prescribed for mobility assistance for patients with mobility impairments. Compared with mobility assistance devices such as wheelchairs, walkers, crutches and scooters, an assistive exosuit may be less bulky, more visually appealing, and conform with activities of daily living such as riding in vehicles, attending community or social functions, using the toilet, and common household activities.

An assistive exosuit may additionally function as primary apparel, fashion items or accessories. The exosuit may be stylized for desired visual appearance. The stylized design may reinforce visual perception of the assistance that the exosuit is intended to provide. For example, an assistive exosuit intended to assist with torso and upper body activities may present a visual appearance of a muscular torso and upper body. Alternatively, the stylized design may be intended to mask or camouflage the functionality of the assistive exosuit through design of the base layer, electro/mechanical integration or other design factors.

Similarly to assistive exosuits intended for medically prescribed mobility assistance, assistive exosuits may be developed and utilized for non-medical mobility assistance, performance enhancement and support. For many, independent aging is associated with greater quality of life, however activities may become more limited with time due to normal aging processes. An assistive exosuit may enable aging individuals living independently to electively enhance their abilities and activities. For example, gait or walking assistance could enable individuals to maintain routines such as social walking or golf. Postural assistance may render social situations more comfortable, with less fatigue. Assistance with transitioning between seated and standing positions may reduce fatigue, increase confidence, and reduce the risk of falls. These types of assistance, while not explicitly medical in nature, may enable more fulfilling, independent living during aging processes.

Athletic applications for an assistive exosuit are also envisioned. In one example, an exosuit may be optimized to assist with a particular activity, such as cycling. In the cycling example, FLAs approximating gluteal or hip extensor muscles may be integrated into bicycle clothing, providing assistance with pedaling. The assistance could be varied based on terrain, fatigue level or strength of the wearer, or other factors. The assistance provided may enable increased performance, injury avoidance, or maintenance of performance in the case of injury or aging. It can be appreciated that assistive exosuits could be optimized to assist with the demands of other sports such as running, jumping, swimming, skiing, or other activities. An athletic assistive exosuit may also be optimized for training in a particular sport or activity. Assistive exosuits may guide the wearer in proper form or technique, such as a golf swing, running stride, skiing form, swimming stroke, or other components of sports or activities. Assistive exosuits may also provide resistance for strength or endurance training. The provided resistance may be according to a regimen, such as high intensity intervals.

Assistive exosuit systems as described above may also be used in gaming applications. Motions of the wearer, detected by the suit, may be incorporated as a game controller system. For example, the suit may sense wearer's motions that simulate running, jumping, throwing, dancing, fighting, or other motions appropriate to a particular game. The suit may provide haptic feedback to the wearer, including resistance or assistance with the motions performed or other haptic feedback to the wearer.

Assistive exosuits as described above may be used for military or first responder applications. Military and first responder personnel are often to be required to perform arduous work where safety or even life may be at stake. An assistive exosuit may provide additional strength or endurance as required for these occupations. An assistive exosuit may connect to one or more communication networks to provide communication services for the wearer, as well as remote monitoring of the suit or wearer.

Assistive exosuits as described above may be used for industrial or occupational safety applications. Exosuits may provide more strength or endurance for specific physical tasks such as lifting or carrying or repetitive tasks such as assembly line work. By providing physical assistance, assistive exosuits may also help avoid or prevent occupational injury due overexertion or repetitive stress.

Assistive exosuits as described above may also be configured as home accessories. Home accessory assistive exosuits may assist with household tasks such as cleaning or yard work, or may be used for recreational or exercise purposes. The communication capabilities of an assistive exosuit may connect to a home network for communication, entertainment or safety monitoring purposes.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art can appreciate that the conception, upon which this disclosure is based, may readily be utilized as a basis for the designing of other structures, systems, methods and media for carrying out the several purposes of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. An exosuit system, comprising:
an exosuit comprising a base layer, a power layer, and a plurality of sensors, wherein the exosuit is operative to provide the plurality of assistive movements; and
control circuitry coupled to the power layer and the plurality of sensors, the control circuitry operative to:
receive data from the plurality of sensors during an exosuit use period;
identify a lift event in the received sensor data;
analyze data associated with the identified lift event to determine quality of the lift event; and
provide feedback via the exosuit based on the determined quality of the lift event.

2. The exosuit system of claim 1, wherein the plurality of sensors are 6-axis inertial measurement units (IMUs), and wherein first, second, and third sensors of the plurality of sensors are located on the torso, left leg, and right leg, respectively, of the exosuit.

3. The exosuit system of claim 2, wherein the first, second, and third sensor provide pitch angles, wherein the control circuitry is operative to:
compare the pitch angles associated with the first sensor to the pitch angles associated with the second and third sensors to determine the quality of the lift event.

4. The exosuit system of claim 3, wherein when relative pitch angles associated with the first sensor are greater than relative pitch angles associated with the second and third sensors, the control circuitry is operative to provide feedback indicative of an unsafe lift event.

5. The exosuit system of claim 3, wherein when relative pitch angles associated with the first sensor are greater than relative pitch angles associated with the second and third sensors, the control circuitry is operative to instruct the exosuit to provide lift assistance.

6. The exosuit system of claim 3, wherein when relative pitch angles associated with the first sensor are less than relative pitch angles associated with the second and third sensors, the control circuitry is operative to provide positive feedback indicative of a safe lift event.

7. An exosuit system, comprising:
an exosuit comprising a base layer, a power layer, and at least one sensor, wherein the exosuit is operative to provide the plurality of assistive movements; and
control circuitry coupled to the power layer and the at least one sensor, the control circuitry operative to:
receive data from the at least one sensor during an exosuit use period, wherein the at least one sensor provides acceleration readings;
identify a lift event in the received sensor data by analyzing acceleration magnitudes obtained from the acceleration readings;
when a lift event is identified, analyze pitch angles associated with the identified lift event to determine quality of the lift event, wherein the pitch angles are derived from the acceleration readings; and
provide feedback via the exosuit based on the determined quality of the lift event.

8. The exosuit system of claim 7, wherein the control circuitry is operative to execute a state machine to positively identify the lift event, wherein the state machine is operative to verify existence of an impulse in the acceleration magnitudes that satisfy lift event criteria.

9. The exosuit system of claim 8, wherein the state machine is operative to reject a potential lift event if any of the lift event criteria fail.

10. The exosuit system of claim 8, wherein the impulse comprises first, second, and third acceleration transitions, wherein the lift event criteria comprises acceleration conditions and timing conditions for each of the first, second, and third acceleration conditions.

11. The exosuit system of claim 10, wherein the state machine is operative to verify that the first acceleration transition satisfies a first lower threshold, that the second acceleration transition satisfies an upper threshold, and that the third acceleration transition satisfies a lower threshold.

12. The exosuit system of claim 10, wherein the state machine is operative to verify that the first acceleration transition satisfies a first timing condition, that the second acceleration transition satisfies a second timing condition, and that the third acceleration transition satisfies a third timing condition, wherein the first, second, and third timing conditions refer to intra transition timing parameters of each of the first, second, and third acceleration transitions.

13. The exosuit system of claim 12, wherein the state machine is operative to verify that a first change from the first acceleration transition to the second acceleration condition satisfies a fourth timing condition and that a second change from the second acceleration condition to the third acceleration condition satisfies a fifth timing condition, wherein the fourth and fifth timing conditions refer to inter transition timing parameters of the first, second, and third acceleration transitions.

14. The exosuit system of claim 7, wherein the control circuitry is operative to determine whether the pitch angles satisfy pitch angle criteria to determine the quality of the lift event.

15. The exosuit system of claim 7, wherein the pitch angle criteria comprises a first range of angles associated with a first transition portion of lift impulse signal, a second range of angles associated with a second transition portion of the lift impulse signal, and third range of angles associate with a third transition portion of the lift impulse signal, wherein the lift event is classified as a safe lift event when at least one pitch angel within the first transition portion falls with the first range of angles, at least one pitch angel within the second transition portion falls with the second range of angles, and at least one pitch angel within the third transition portion falls with the third range of angles.

16. The exosuit system of claim 7, wherein the pitch angle criteria comprises reference angles, wherein the pitch angles are compared to the reference angles to determine the quality of the lift event.

17. The exosuit system of claim 7, wherein the control circuitry is operative to instruct the exosuit to provide lift assistance to future lift events in response to detection of an unsafe lift event.

18. The exosuit system of claim 7, wherein the control circuitry is operative to provide feedback in response to detection of an unsafe lift event.
